(19) United States
(12) Patent Application Publication
(10) Pub. No.: US 2015/0157692 A1
(43) Pub. Date: Jun. 11, 2015

(54) METHODS OF TREATING OBESITY

(71) Applicant: The University of Chicago, Chicago, IL (US)

(72) Inventor: Yang-Xin Fu, Chicago, IL (US)

(21) Appl. No.: 14/403,017

(22) PCT Filed: May 23, 2013
(86) PCT No.: PCT/US2013/042478

§ 371 Date: Nov. 21, 2014

Related U.S. Application Data
(60) Provisional application No. 61/650,867, filed on May 23, 2012.

(51) Int. Cl. A61K 38/20; A23L 1/30; C12N 15/11; A23L 1/29; A61K 38/19; C07K 16/24

(52) U.S. Cl. CPC A61K 38/20; A61K 38/191; C07K 16/242; C07K 16/244; C12N 15/11; A23L 1/293; A23L 1/3014; C07K 2317/55; C07K 2317/54; C07K 2317/622; C12N 2310/14; A23V 2002/00

(57) ABSTRACT

The present invention relates to methods of treating and/or preventing obesity comprising the administration of an inhibitor of lymphotoxin, IL-22 and/or IL-23 to a subject having or at risk of developing obesity.

### CROSS REFERENCE TO RELATED APPLICATION

This application claims the benefit of priority to U.S. Provisional Patent Application Ser. No. 61/650,867, filed May 23, 2013, hereby incorporated by reference in its entirety.

This invention was made with government support under grants AI090392 and CA134563 awarded by the National Institutes of Health. The government has certain rights in the invention.

### BACKGROUND

I. Field of the Invention

The present invention relates generally to the fields of biology and medicine. More particularly, it concerns methods for the prevention and/or treatment of obesity.

II. Description of Related Art

Obesity has become a major health problem in the United States and other developed nations. In the United States, 65% of the adult population is considered overweight or obese, and more than 30% of adults meet the criteria for obesity. The World Health Organization has estimated that more than 1 billion adults worldwide are overweight, with 300 million of these considered clinically obese. The incidence of obesity in children is also growing rapidly in many countries. Obesity is a major risk factor for cardiovascular disease, stroke, insulin resistance, type 2 diabetes, liver disease, neurodegenerative disease, respiratory diseases and other severe illnesses, and has been implicated as a risk factor for certain types of cancer including breast and colon cancer. Aside from its impacts on physical health, obesity has significant adverse effects on quality of life and psychological well-being. The incidence of obesity, already high, is likely to grow as a result of increasingly sedentary lifestyles in many countries. In addition, certain widely used psychiatric drugs, notably atypical antipsychotics, are associated with weight gain and increased risk of diabetes. Since these drugs must be used chronically to achieve adequate control of psychiatric symptoms, and treatment compliance in patients with mental disorders is frequently poor, these side effects present both a barrier to compliance and a significant additional health risk to patients.

Although it is well established that weight loss can be achieved through reduced caloric intake and increased physical activity, obesity has continued to be an intractable problem in Western countries, especially in the United States. The discovery of safe and effective drugs to induce weight loss has been a major research goal for decades. However, to date the drugs that have shown efficacy have been burdened with significant side effects or have shown only modest efficacy. For example, amphetamines have been used effectively as appetite suppressants but have a strong risk of dependence along with other side effects. The discovery of leptin, a peptide hormone that plays a major role in appetite regulation, was considered to be a potential breakthrough in the treatment of obesity, but in clinical trials, leptin was not effective. More recently, cannabinoid receptor antagonists were under development as anti-obesity drugs but showed unacceptable psychiatric side effects. Similarly, drugs designed to reduce fat absorption in the digestive tract have been associated with significant gastrointestinal side effects.

Accordingly, there is a significant need for new anti-obesity treatments. In particular, there is a need for anti-obesity treatments with limited side effects that may be safely used in combination with other drugs that are in common use in obese patients, such as antidiabetic drugs, antihypertensive drugs, cholesterol-reducing agents, and insulin. Thus, agents that can be used for the prevention and treatment of obesity would represent a significant advance.

### SUMMARY OF THE INVENTION

Thus, in accordance with the present invention, there are provided a method of stabilizing or reducing weight in a subject in need thereof comprising orally administering to the subject an agent that inhibits lymphotoxin, IL-22 and/or IL-23 expression and/or function in an amount sufficient to stabilize or reduce the subject's weight. The subject may be excess body fat and/or be overweight. The subject's body mass index (BMI) may be from 25 kg/m² to 30 kg/m². The subject may be obese or exhibits one of more symptoms of obesity. The obesity may be class I, II or III. The subject's BMI may be from 30 kg/m² to 35 kg/m², from 35 kg/m² to 40 kg/m², or from 40 kg/m² to 80 kg/m². In some embodiments, a subject's BMI index is at most or at least 30, 35, 40, 45, 50, 55, 60, 65, 70, 75, 80, 85 or more kg/m² (or any range derivable therein). Stabilizing a subject's weight means the patient's weight is maintained within or up to about 0.1, 0.2, 0.3, 0.4, or 0.5% (or any range derivable therein) from the previous week. It is also contemplated that the subject may experience a reduction in weight that is about at least or at most 0.5, 1, 2, 3, 4, 5, 6, 7, 8, 9, 10, 11, 12, 13, 14, 15, 16, 17 18, 19, 20 pounds of more in a week or month (or any range derivable therein). Alternatively, a reduction in weight may be a reduction that is within or up to about 0.05, 0.06, 0.07, 0.08, 0.09, 0.10, 0.20, 0.30, 0.40, 0.50, 0.6, 0.7, 0.8, 0.9, 1, 2, 3, 4, 5, 6, 7, 8, 9, or 10% in a week or month (or any range derivable therein).

The subject may be a human subject. The method may further comprise feeding said subject a low fat and/or low calorie diet. In certain embodiments the patient is on a very low calorie diet (800 kilocalories or less/day). The inhibitor is a small molecule, and antibody, a peptide, or a nucleic acid. The peptide may comprises an inactive fragment of lymphotoxin, IL-22 or IL-23, or an inactive fragment of a lymphotoxin receptor, a IL-22 receptor or a IL-23 receptor. The antibody may bind to a domain on lymphotoxin, IL-22 or IL-23 that interacts with the cognate receptor. The nucleic acid may be an single-stranded or double-stranded inhibitory oligonucleotide for lymphotoxin, IL-22 or IL-23.

The agent may administered daily. The agent may be formulated as a probiotic foodstuff. The weight of the subject may have been measured or will be measured. The weight of the subject may have been measured prior to administering the agent and will be measured after administering the agent. The BMI of the subject may have been measured or will be measured. The BMI of the subject may have been measured prior to administering the agent and will be measured after administering the agent. The method may further comprise assessing lymphotoxin and/or IL-22 and/or IL-23 expression or levels in a sample from said subject. The sample may be a stool sample.

Also provided is a method of preventing or inhibiting weight gain in a subject in need thereof comprising orally administering to the subject an agent that inhibits lymphotoxin, IL-22 and/or IL-23 expression and/or function in an amount sufficient to prevent or inhibit and increase in the subject's weight. The subject may be a human subject. The method may further comprise feeding said subject a low fat and/or low calorie diet. In certain embodiments the patient is on a very low calorie diet (800 kilocalories or less/day). The agent may administered daily. The agent may be formulated as a probiotic foodstuff.

The inhibitor is a small molecule, and antibody, a peptide, or a nucleic acid. The peptide may comprises an inactive fragment of lymphotoxin, IL-22 or IL-23, or an inactive fragment of a lymphotoxin receptor, a IL-22 receptor or a IL-23 receptor. The antibody may bind to a domain on lymphotoxin, IL-22 or IL-23 that interacts with the cognate receptor. The nucleic acid may be an single-stranded or double-stranded inhibitory oligonucleotide for lymphotoxin, IL-22 or IL-23.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The invention may be better understood by reference to one of these drawings in combination with the detailed description of specific embodiments presented herein.

FIGS. 1A-F: LTβR is essential for weight gain in response to HFD. (A-F) 9 week old WT (C57BL6), LTβR^{-/-}, or LTα^{-/-} animals were subject to a HFD or NCD for 9 weeks. (A) Weight gain as a percentage of starting weight is plotted (B) Absolute weight in grams at the end of diet. (C) Representative mice from WT HFD and LTβR^{-/-} HFD groups at time of sacrifice (WT nearest ruler). (D) Perigonadal fat was removed and weighed at the end of diet; weight of fat is plotted. (E) Weight gain as a percentage of starting weight is plotted against days on diet. (F) Weight at the end of diet for mice in E. (n=5-12 mice per group; growth curve is reflective of 3 independent experiments and statistics demonstrate differences between HFD groups; Student's t-test for individual points along growth curves; 1-Way Anova for dot plots with Bonferonni post-test: *p<0.05, **p<0.01, ***p<0.001)

FIGS. 2A-D: LTβR influences weight gain through changes in the microbiota. (A) Food was measured in cages mice and the difference in food between measurements is plotted (data is representative of 3 independent experiments), (B-C) Germ free mice were gavaged with cecal contents from LTβR^{-/-} or LTβR^{-/-} littermates maintained on NCD or HFD for 9 weeks starting at 9 weeks of age. Cecal contents from two donors was pooled. Recipients were kept on diets of similar compositions to donors. (B-C) Weight gain as a percentage of starting body weight is shown 20 days after gavage of germ free recipients from the NCD (B) and HFD (C) groups. (D) RT-PCR for SFB on DNA from stool collected from LTβR^{+/-} and LTβR^{-/-} mice 4 weeks after NCD start (n=4 mice per group, representative of 3 independent experiments). (n=3-5 germ free mice/group, representative of 2 independent experiments Summary of p-values: *p<0.05, **p<0.01, ***p<0.001, student's t test for a and d, paired-t test for b and c).

FIGS. 3A-C: Environmental exposure reveal horizontal transmissibility of the obese phenotype. (A-C) LTβR^{+/-} or LTβR^{-/-} were genotyped and weaned either separately or together (Cohouse) at 3 weeks of age. (A) Weight gain as a percentage of starting weight is plotted. (B) Weight gain after 9 weeks of diet. (C) RT-PCR for STB in stool relative to LTβR^{+/-} littermates stool at diet start. (n=5-12 mice per group; growth curve is reflective of 3 independent experiments and statistics demonstrate differences between HFD groups; Student's t-test for individual points along growth curves and dot-plots: *p<0.05, **p<0.01, ***p<0.001).

FIGS. 4A-H: LTβR agonizes the innate IL-23/IL-22 axis. LTβR^{+/-} and LTβR^{-/-} animals were fed HFD. After challenge, PCR for targets was performed, (A, B, D, E) There were not significant changes in expression for TGFβ, IL-6, IL-17A, or IL-17F. (C, F-H) There were significant differences between groups for in IL-23, IL-22, RegIIIγ, and RegIIIβ: (n=3-9 mice per group, *p<0.05, **p<0.01, student's t test).

FIGS. 5A-D: HFD induces LTβR-dependent IL-23 which is essential for DIO. (A) WT (C57BL/6), LTβR^{-/-}, and LTβR^{-/-} animals were fed HFD for 10 weeks. At the end of diet, animals were sacrificed and colons were removed and cultured overnight. Supernatants were subjected to ELISA for IL-23p19/p40, (B-D) WT (C57BL/6) mice or p19^{-/-} animals were challenged with HFD starting at 9 weeks of age or 9 weeks. (B) Weight gain as a percentage of starting weight is plotted. (C) Perigonadal fat was removed and weighed at the end of diet; weight of fat is plotted. (D) Fat from (C) is plotted as a percentage of body weight. Weight gain as a percentage of starting weight is plotted. (n=5-9 mice per group; growth curve is reflective of 2 independent experiments and statistics demonstrate differences between 1-IM groups; Student's t-test for individual points along growth curves; 1-Way Anova for dot plots with Bonferonni post-test: *p<0.03. **p<0.01, ***p<0.001).

FIGS. 6A-D: The transcription factor, RORγt, is required for weight gain and SFB homeostasis in DIO. RORγt^{+/-} or RORγt^{-/-} littermates were challenged with HFD for 9 weeks starting at 5 weeks of age. (A) Weight gain as a percentage of starting weight is plotted. (B) RT-PCR for SFB in stool relative to RORγt^{+/-} littermates at diet start (n=4 mice in each group). (C) Perigonadal fat was removed and weighed at the end of diet; weight of fat is plotted. (D) Fat from (C) is plotted as a percentage of body weight. Weight gain as a percentage of starting weight is plotted (n=7-8 mice per group; growth curve is reflective of 3 independent experiments and statistics demonstrate differences between HFD groups; Student's t-test for individual points along growth curves and SFB levels; 1-Way Anova for dot plots with Bonferonni post-test: *p<0.05, **p<0.01, ***p<0.001).

FIGS. 7A-C: LTβR^{-/-}, or LTα^{-/-} mice resist to fat accumulation in response to HFD. 9 week old WT (C57BL6), LTβR^{-/-}, or LTα^{-/-} animals were subject to a HFD or NCD for 9 weeks, Perigonadal fat was removed and weighed at the end of diet; weight of fat is plotted as a percentage of body weight (A, C) or in grams (B). (n=7-12 mice per group, growth curve is reflective of 3 independent experiments, statistics demonstrate differences between HFD groups: *p<0.05, **p<0.01, ***p<0.001, One-way Analysis of Variance with Bonferonni post-test).

FIGS. 8A-C: LTβ mice also resist to weight gain and fat accumulation in response to HFD. 9 week-old WT (LTβf/f animals) or LTβ were subject to a HFD for 9 weeks, (B) Absolute weight in grams is plotted against days on diet. (B) Weight gain as a percentage of starting weight is plotted. (C) Perigonadal fat was removed and weighed at the end of diet; weight of fat is plotted (A-B, Two-way Analysis of Variance with Bonferonni post-test; C, student's t test; *p<0.05, **p<0.01, ***p<0.001).

FIG. 9: LTβR is Essential for Commensal Microbiota homeostasis. Principle coordinate analysis of V1-V2 tags from LTβR^{+/-} and LTβR^{-/-} cecal contents after HFD was performed using Mothur, and clustering by two largest determinants of variation is shown.

FIG. 10A-10B: HFD induces Expansion of Firmicute phyla members in both groups and reduces Commensal Diversity in LTβR Sufficient Animals. V1-V2 tags from LTβR^{+/-} and LTβR^{-/-} stool DNA at 0 wk HFD (while NCD) and 4 weeks after the start of HFD were subjected to 454 Pyrosequencing. (A) Composition at the phyla level is shown for each genotype and diet. (B) Simpson-Diversity Index values were calculated for each sample (higher index values indicate lower diversity; student's t test, *p<0.05,

FIG. 11: Candidate OTU GXPY6ZS16J2P5K alignment with V1-V2 region of known V1-V2 region of SFB 16s rDNA. GXPY6ZS16J2P5K was one of 76 unclassified Clostridiales order members that were sequenced. GXPY6ZS16J2P5K was chosen as a representative.

FIG. 12: SFB overgrowth occurs within the Ileum. DNA was extracted from ileal scrapings after 10 weeks of HFD. RT-PCR for SFB was performed. Statistics reflect two-tailed Mann-Whitney t-test between groups.

FIG. 13: LTβR agonizes the IL-23/IL-22 axis (NCD and WT Supplement). WT(C57BL6), LTβR^{+/-} and LTβR^{-/-} animals were fed HFD or NCD (LTβR^{+/-} and LTβR^{-/-} only). After challenge, PCR for targets was performed. Significant differences are indicated (n=3-9 mice per group, *p<0.05, p<0.01, Bonferonni post test after 1-Way ANOVA).

FIGS. 14A-14C: IL-22 restore's SUB clearance and perigonadal fat pad expansion in LTβR^{-/-} animals. WT(C57BL/6) or LTβR^{-/-} animals received 10 μg of empty vector (pERK) or vector encoding IL-22 (IL22) by hydrodynamic injection at day −2 of diet. (A) SFB levels were determined by RT-PCR. (B-C) Perigonadal fat pads were dissected at the end of diet and plotted in absolute terms (B) or as a percentage of total body weight (C) (*p<0.05, student's t test).

### DESCRIPTION OF ILLUSTRATIVE EMBODIMENTS

The current invention provides methods of using inhibitors of the lymphotoxin/IL-22/IL-23 signaling axis to induce weight loss or prevent weight gain/obesity in patients having established obesity and complications thereof, and or at risk of developing the same. These and other aspects of the invention are described in greater detail below.

### I. OBESITY

The present disclosure concerns new methods for the treatment and prevention of obesity. Obesity is a medical condition in which excess body fat has accumulated to the extent that it may have an adverse effect on health. It is typically defined by body mass index (BMI) and may be further evaluated in terms of fat distribution via the waist hip ratio and total cardiovascular risk factors. BMI is related to both percentage body fat and total body fat.

BMI is calculated by dividing the subject's mass by the square of his or her height (in metric units: kilograms/meters²). The definitions established by the World Health Organization (WHO) in 1997 and published in 2000 are listed below:

| BMI | Classification |
|---|---|
| <18.5 | underweight |
| 18.5-24.9 | normal weight |
| 25.0-29.9 | overweight |
| 30.0-34.9 | class I obesity |
| 35.0-39.9 | class II obesity |
| ≧40.0 | class III obesity |

Obesity increases the risk of many physical and mental conditions. These comorbidities are most commonly shown in metabolic syndrome, a combination of medical disorders which includes: diabetes mellitus type 2, high blood pressure, high blood cholesterol, and high triglyceride levels.

A substantial body of research supports an association between obesity and a chronic, “smoldering” inflammatory state. Obesity is associated with overproduction of inflammatory cytokines and chronic activation of inflammatory signaling pathways, including the NF-kB pathway (Hotamisligil, 2006). Chronic inflammation in adipose tissue is linked with the development of insulin resistance in skeletal muscle (Guilherme et al., 2008). Chronic activation of the NF-κB pathway has been shown to induce insulin resistance and NF-κB inhibition has been proposed as a therapeutic strategy for the treatment of Type 2 diabetes (Arkan et al., 2005; Shoelson et al., 2006).

In a fashion analogous to the development of insulin resistance, obesity has been associated with the development of resistance to the action of leptin. Leptin, a peptide hormone, has complex biological effects but one important site of action is the mediobasal hypothalamus. This structure of the brain is known to exert control over feeding behavior and energy homeostasis. Recently, oxidative stress and activation of the NF-κB pathway in the hypothalamus were shown to be linked to hypothalamic insulin and leptin resistance (Zhang et al., 2008). Activation of the antioxidant transcription factor Nrf2 is known to inhibit NF-κB activity, and Nrf2 activation by a semisynthetic triterpenoid has been reported to inhibit the development of obesity in mice fed on a high-fat diet (Shin et al., 2009).

### II. OBESITY TARGETS

A. Lymphotoxin

Lymphotoxin α, previously known as tumor necrosis factor-β, is a lymphokine cytokine. It is a protein that is produced by Th1 type T-cells and induces vascular endothelial cells to change their surface adhesion molecules to allow phagocytic cells to bind to them. Lymphotoxin is homologous to Tumor Necrosis Factor beta, but secreted by T-cells. It is paracrine due to the small amounts produced. The effects are similar to TNF-α, but TNF-β is also important for the development of lymphoid organs.

The accession numbers for the human mRNA sequence is NM_{—}000595.

B. IL-22

Interleukin-22 (IL-22) is protein that in humans is encoded by the 11.22 gene. IL-22 a member of a group of cytokines called the IL-10 family or IL-10 superfamily (including IL-19, IL-20, IL-24, and IL-26), a class of potent mediators of cellular inflammatory responses. It shares use of IL-10R2 in cell signaling with other members of this family, IL-10, IL-26, IL-28A/B and IL-29. IL-22 is produced by activated DC and T cells and initiates innate immune responses against bacterial pathogens especially in epithelial cells such as respiratory and gut epithelial cells. IL-22 along with IL-17 is rapidly produced by splenic LTi-like cells and can be also produced by Th17 cells and likely plays a role in the coordinated response of both adaptive and innate immune systems.

IL-22 biological activity is initiated by binding to a cell-surface complex composed of IL-22R1 and IL-10R2 receptor chains and further regulated by interactions with a soluble binding protein, IL-22BP, which shares sequence similarity with an extracellular region of IL-22R1 (sIL-22R1). IL-22 and IL-10 receptor chains play a role in cellular targeting and signal transduction to selectively initiate and regulate immune responses IL-22 can contribute to immune disease through the stimulation of inflammatory responses, S100s and defensins. IL-22 also promotes hepatocyte survival in the liver and epithelial cells in the lung and gut similar to IL-10. In some contexts, the pro-inflammatory versus tissue-protective functions of IL-22 are regulated by the often co-expressed cytokine IL-17.

IL-22 is an α-helical cytokine. IL-22 binds to a heterodimeric cell surface receptor composed of IL-10R2 and IL-22R1 subunits. Crystalization is possible if the N-linked glycosylation sites are removed in mutants of IL-22 bound with high-affinity cell-surface receptor sIL-22R1. The crystallographic asymmetric unit contained two IL-22-sIL-22R1 complexes.

IL-22, signals through the interferon receptor-related proteins CRF2-4 and IL-22R. It forms cell surface complexes with IL-22R1 and IL-10R2 chains resulting in signal transduction through receptor, IL-10R2. The IL-22/IL-22R1/IL-10R2 complex activates intracellular kinases (JAK1, Tyk2, and MAP kinases) and transcription factors, especially STAT3. It can induce IL-20 and IL-24 signaling when IL-22R1 pairs with IL-20R2.

The accession numbers for human mRNA and protein sequences are NM_{—}020525.4 and NP_{—}065386.1, respectively.

C. IL-23

Interleukin-23 subunit alpha is a protein that in humans is encoded by the IL23A gene. IL-23 is produced by dendritic cells and macrophages. Moreover, IL-23 is stimulated by Danger Signals, including cell debris, and directs memory T cells to Th17 response. This gene encodes the p19 subunit of the heterodimeric cytokine interleukin 23 (IL23). IL23 is composed of this protein and the p40 subunit of interleukin 12 (IL12B). The receptor of IL23 is formed by the beta 1 subunit of IL12 (IL12RB1) and an IL23 specific subunit, IL23R. Both IL23 and IL12 can activate the transcription activator STAT4, and stimulate the production of interferon-gamma (IFNG). In contrast to IL12, which acts mainly on naive CD4(+) T cells, IL23 preferentially acts on memory CD4(+) T cells.

Interleukin-23 (IL-23) is a heterodimeric cytokine consisting of two subunits, one called p40, which is shared with another cytokine, IL-12, and another called p19 (the IL-23 alpha subunit). In other words, IL-23 is a dimer of p40-S-S-p19. IL-23 is an important part of the inflammatory response against infection. It promotes upregulation of the matrix metalloprotease MMP9, increases angiogenesis and reduces CD8+T-cell infiltration. Recently, IL-23 has been implicated in the development of cancerous tumors. In conjunction with IL-6 and TGF-β1, IL-23 stimulates naive CD4+T cells to differentiate into a novel subset of cells called Th17 cells, which are distinct from the classical Th1 and Th2 cells. Th17 cells produce IL-17, a proinflammatory cytokine that enhances T cell priming and stimulates the production of proinflammatory molecules such as IL-1, IL-6, TNT-alpha, NOS-2, and chemokines resulting in inflammation. Knockout mice deficient in either p40 or p19, or in either subunit of the IL-23 receptor (IL-23R and IL12R-β1) develop less severe symptoms of multiple sclerosis and inflammatory bowel disease highlighting the importance of IL-23 in the inflammatory pathway.

The accession number for the human mRNA and protein sequences are NM_{—}016584.2 and NP_{—}057668.1, respectively.

### III. INHIBITORS, PHARMACEUTICAL FORMULATIONS AND ROUTES OF ADMINISTRATION

As discussed above, the present invention contemplates the prevention, treatment or inhibition of obesity development or progression using inhibitors of lymphotoxin, IL-22 and/or IL-23, which the inventor has identified as playing a role in inflammatory signaling leading to weight gain. A number of inhibitors for these molecules currently exist. Lymphotoxin inhibitors include Baminercept Alfa (Biogen; a.k.a. BG9924 and LTβR-Ig) that binds to the LTBR and LIGHT ligands (world-wide-web at medicalnewstoday.com/releases/111181.php; incorporated by reference), Etanercept (a.k.a. Enbrel: Gudbrandsdottir, Clin Exp Rheumatol. 22(1):118-24. 2004; incorporated by reference) and soluble lymphotoxin receptor (U.S. Patent Publication 2008/0219967. IL-22 inhibitors include the IL-22 specific mAb Fezakinumab (a.k.a. ILV-094 (Pfizer)) and the IL-22 binding protein (IL-22BP; Weber, Infection Immun. 75:1690-1697, 2007; incorporated by reference), which is a recombinant IL-2213P with a with a noncytolytic Fc[1]2a fragment. IL-23 inhibitors including STA-5326, aka apilimod mesylate (Synta. Pharmaceuticals; Keino et al., Arthritis Res. Ther. 10:1-8, 2008; incorporated by reference), Ustekinumab (a.k.a. CNTO-1275), which is a mAb that inhibits the p40 subunits common to IL-12 and IL-23 (Yeilding, Ann. NY Acad. Sci. 122:30-39, 2011; incorporated by reference), Briakinumab (ABT-874), SCH 900222 mAb (Merck), which targets the p19 subunit of IL-23, and CNTO (Centocor), which targets the p19 subunit of IL-23.

The following provides a more general discussion of such inhibitors and their use as monotherapies or in combination with other agents designed to target either causative microbiota in the subject's gut, or these same targets.

A. Inhibitors of LT, IL-22 and IL-23

i. Antisense Constructs

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA, Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

ii. Ribozymes

Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cook, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cook et al., 1981; Michel and Westhof, 1990; Reinhold-Hurek and Shub, 1992). This specificity has been attributed to the requirement that the substrate bind via specific base-pairing interactions to the internal guide sequence (“MS”) of the ribozyme prior to chemical reaction.

Ribozyme catalysis has primarily been observed as part of sequence-specific cleavage/ligation reactions involving nucleic acids (Joyce, 1989; Cook et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HINT Most of this work involved the modification of a target mRNA, based on a specific mutant codon that is cleaved by a specific ribozyme

iii. RNAi

RNA interference (also referred to as “RNA-mediated interference” or RNAi) is a mechanism by which gene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin and Avery et al., 1999; Montgomery et al., 1998; Sharp and Zamore, 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al, 2000; Ketting et al., 1999; Lin and Avery et al., 1999; Montgomery et al., 1998; Sharp et al., 1999; Sharp and Zamore. 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp et al., 1999; Sharp and Zamore, 2000; Elbashir et al., 2001). It is generally accepted that RNAi acts post-transcriptionally, targeting RNA transcripts for degradation. It appears that both nuclear and cytoplasmic RNA can be targeted (Bosher and Labouesse, 2000).

siRNAs must be designed so that they are specific and effective in suppressing the expression of the genes of interest. Methods of selecting the target sequences, i.e., those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

The making of siRNAs has been mainly through direct chemical synthesis; through processing of longer, double stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single stranded RNA-oligomers followed by the annealing of the two single stranded oligomers into a double stranded RNA. Methods of chemical synthesis are diverse. Non-limiting examples are provided in U.S. Pat. Nos. 5,889,136, 4,415,723, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3 non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically synthesized siRNAs are found to work optimally when they are in cell culture at concentrations of 25-100 nM, but concentrations of about 100 nM have achieved effective suppression of expression in mammalian cells. siRNAs have been most effective in mammalian cell culture at about 100 nM. In several instances, however, lower concentrations of chemically synthesized siRNA have been used (Caplen, et al., 2000; Elbashir et al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25 mer lengths with the endogenous nuclease complex that converts long dsRNAs to siRNAs in vivo. They do not describe or present data for synthesizing and using in vitro transcribed 21-25 mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single-stranded RNA is enzymatically synthesized from the PCR products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

iv. Peptides and Polypeptide Fragments

In accordance with the present invention, one can provide competitive and/or non-functional fragments or portions of lymphotoxin, IL-22 and/or IL-23 or their cognate receptors. In the former, the concept would be to inhibit signaling of native lymphotoxin, IL-22 and IL-23 by binding up the receptors with molecules that interact, but fail to activate those receptors. In the latter, the opposite approach is employed, i.e., using soluble non-functional receptor to “soak up” circulating lymphotoxin, IL-22 and IL-23, thereby preventing it from signaling through the native receptor.

Thus, the present invention contemplates the design, production and use of various peptides. In general, peptides will be 50 residues or less, while polypeptide fragments may be larger. The present invention may utilize L-configuration amino acids, D-configuration amino acids, or a mixture thereof. While L-amino acids represent the vast majority of amino acids found in proteins, D-amino acids are found in some proteins produced by exotic sea-dwelling organisms, such as cone snails. They are also abundant components of the peptidoglycan cell walls of bacteria. D-serine may act as a neurotransmitter in the brain. The L and D convention for amino acid configuration refers not to the optical activity of the amino acid itself, but rather to the optical activity of the isomer of glyceraldehyde from which that amino acid can theoretically be synthesized (D-glyceraldehyde is dextrorotary; L-glyceraldehyde is levorotary).

One form of an “all-D” peptide is a retro-inverso peptide. Retro-inverso modification of naturally occurring polypeptides involves the synthetic assemblage of amino acids with α-carbon stereochemistry opposite to that of the corresponding L-amino acids, i.e., D-amino acids in reverse order with respect to the native peptide sequence. A retro-inverso analogue thus has reversed termini and reversed direction of peptide bonds (NH—CO rather than CO—NH) while approximately maintaining the topology of the side chains as in the native peptide sequence. See U.S. Pat. No. 6,261,569, incorporated herein by reference.

Peptides may be modified for in vivo use by the addition, at the amino- and/or carboxyl-terminal ends, of a blocking agent to facilitate survival of the peptide in vivo are contemplated. This can be useful in those situations in which the peptide termini tend to be degraded by proteases prior to cellular uptake. Such blocking agents can include, without limitation, additional related or unrelated peptide sequences that can be attached to the amino and/or carboxyl terminal residues of the peptide to be administered. These agents can be added either chemically during the synthesis of the peptide, or by recombinant DNA technology by methods familiar in the art. Alternatively, blocking agents such as pyroglutamic acid or other molecules known in the art can be attached to the amino and/or carboxyl terminal residues.

It will be advantageous to produce peptides using the solid-phase synthetic techniques (Merrifield, 1963). Other peptide synthesis techniques are well known to those of skill in the art (Bodanszky et al, 1976; Peptide Synthesis, 1985; Solid Phase Peptide Synthelia, 1984). Appropriate protective groups for use in such syntheses will be found in the above texts, as well as in Protective Groups in Organic Chemistry, 1973. These synthetic methods involve the sequential addition of one or more amino acid residues or suitable protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group, such as lysine.

Using solid phase synthesis as an example, the protected or derivatized amino acid is attached to an inert solid support through its unprotected carboxyl or amino group. The protecting group of the amino or carboxyl group is then selectively removed and the next amino acid in the sequence having the complementary (amino or carboxyl) group suitably protected is admixed and reacted with the residue already attached to the solid support. The protecting group of the amino or carboxyl group is then removed from this newly added amino acid residue, and the next amino acid (suitably protected) is then added, and so forth. After all the desired amino acids have been linked in the proper sequence, any remaining terminal and side group protecting groups (and solid support) are removed sequentially or concurrently, to provide the final peptide. The peptides of the invention are preferably devoid of benzylated or methylbenzylated amino acids. Such protecting group moieties may be used in the course of synthesis, but they are removed before the peptides are used. Additional reactions may be necessary, as described elsewhere, to form intramolecular linkages to restrain conformation.

v. Antibodies

Antibodies according to the present invention are those that bind to and inhibit the pro-inflammatory functions of lymphotoxin, IL-22 and IL-23. Antibodies of this nature are available commercially and can be made by techniques well known to those of skill in the art. Such antibodies may be defined by their binding specificity, their activity (inhibition) profile, or by their sequence (e.g., CDRs).

Antibodies can be categorized by Ig class. IgM's are the primary antibodies against A and B antigens on red blood cells. IgM is by far the physically largest antibody in the human circulatory system. It is the first antibody to appear in response to initial exposure to antigen. IgM forms polymers where multiple immunoglobulins are covalently linked together with disulfide bonds, mostly as a pentamer but also as a hexamer. IgM has a molecular mass of approximately 900 kDa (in its pentamer form). Because each monomer has two antigen binding sites, a pentameric IgM has 10 binding sites. Typically, however, IgM cannot bind 10 antigens at the same time because the large size of most antigens hinders binding to nearby sites.

The J chain is found in pentameric IgM but not in the hexameric form, perhaps due to space constraints in the hexameric complex. Pentameric IgM can also be made in the absence of J chain. At present, it is still uncertain what fraction of normal pentamer contains J chain, and to this extent it is also uncertain whether a J chain-containing pentamer contains one or more than one J chain.

Because IgM is a large molecule, it cannot diffuse well, and is found in the interstitium only in very low quantities. IgM is primarily found in serum; however, because of the J chain, it is also important as a secretory immunoglobulin. However, due to its polymeric nature, IgM possesses high avidity, and is particularly effective at complement activation. By itself, IgM is an ineffective opsonin; however it contributes greatly to opsonization by activating complement and causing C3b to bind to the antigen.

In germline cells, the gene segment encoding the μ constant region of the heavy chain is positioned first among other constant region gene segments. For this reason, IgM is the first immunoglobulin expressed by mature B cells. It is also the first immunoglobulin expressed in the fetus (around 20 weeks) and also phylogenetically the earliest antibody to develop. IgM antibodies appear early in the course of an infection and usually reappear, to a lesser extent, after further exposure. IgM antibodies do not pass across the human placenta (only isotype IgG). These two biological properties of IgM make it useful in the diagnosis of infectious diseases. Demonstrating IgM antibodies in a patient's serum indicates recent infection, or in a neonate's serum indicates intrauterine infection (e.g., congenital rubella).

IgM in normal serum is often found to bind to specific antigens, even in the absence of prior immunization. For this reason IgM has sometimes been called a “natural antibody.” This phenomenon is probably due to the high avidity of IgM that allow it to bind detectably even to weakly cross-reacting antigens that are naturally occurring. For example the IgM antibodies that bind to the red blood cell A and B antigens might be formed in early life as a result of exposure to A- and B-like substances that are present on bacteria or perhaps also on plant materials. IgM antibodies are mainly responsible for the clumping (agglutination) of red blood cells if the recipient of a blood transfusion receives blood that is not compatible with their blood type.

IgM is more sensitive to denaturation by 2-mercaptoethanol than IgG. This technique was historically used to distinguish between these isotypes before specific anti-IgG and anti-IgM secondary antibodies for immunoassays became commercially available. Serum samples would be tested for reactivity with an antigen before or after 2-mercaptoethanol treatment to determine whether the activity was due to IgM or IgG.

In various embodiments, one may choose to engineer sequences of the identified antibodies for a variety of reasons, such as improved expression, improved cross-reactivity, diminished off-target binding or abrogation of one or more natural effector functions, such as activation of complement or recruitment of immune cells (e.g., T In particular, IgM antibodies may be converted to IgG antibodies. The following is a general discussion of relevant techniques for antibody engineering.

Hybridomas (produced according to standard metholodies) may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns. Recombinant full length IgG antibodies can be generated by subcloning heavy and light chain iv DNAs from the cloning vector into a Lonza pConIgG1 or pConK2 plasmid vector, transfected into 293 Freestyle cells or Lonza CHO cells, and collected and purified from the CHO cell supernatant.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

pCon Vectors™ are an easy way to re-express whole antibodies. The constant region vectors are a set of vectors offering a range of immunoglobulin constant region vectors cloned into the pEE vectors. These vectors offer easy construction of full length antibodies with human constant regions and the convenience of the GS System™.

Antibody molecules will comprise fragments (such as F(ab′), F(ab′)₂) that are produced, for example, by the proteolytic cleavage of the mAbs, or single-chain immunoglobulins producible, for example, via recombinant means. Such antibody derivatives are monovalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

It may be desirable to “humanize” antibodies produced in non-human hosts in order to attenuate any immune reaction when used in human therapy. Such humanized antibodies may be studied in an in vitro or an in vivo context. Humanized antibodies may be produced, for example by replacing an immunogenic portion of an antibody with a corresponding, but non-immunogenic portion (i.e., chimeric antibodies). PCT Application PCT/US86/02269 EP Application 184,187; EP Application 171,496; EP Application 173,494; PCT Application WO 86/01533; EP Application 125,023; Sun et al. (1987); Wood et al. (1985); and Shaw et at (1988); all of which references are incorporated herein by reference. General reviews of “humanized” chimeric antibodies are provided by Morrison (1985); also incorporated herein by reference. “Humanized” antibodies can alternatively be produced by CDR or CEA substitution. Jones et al. (1986); Verhoeyen et al. (1988); Beidler et al. (1988); all of which are incorporated herein by reference.

In related embodiments, the antibody is a derivative of the disclosed antibodies, e.g., an antibody comprising the CDR sequences identical to those in the disclosed antibodies (e.g., a chimeric, humanized or CDR-grafted antibody). In yet a further embodiment, the antibody is a fully human recombinant antibody.

The present invention also contemplates isotype modification. By modifying the Fc region to have a different isotype, different functionalities can be achieved. For example, changing to IgG₄ can reduce immune effector functions associated with other isotypes.

Modified antibodies may be made by any technique known to those of skill in the art, including expression through standard molecular biological techniques, or the chemical synthesis of polypeptides. Methods for recombinant expression are addressed elsewhere in this document. For example, a Single Chain Variable Fragment (scFv) is a fusion of the variable regions of the heavy and light chains of immunoglobulins, linked together with a short (usually serine, glycine) linker. This chimeric molecule, also known as a single domain antibody, retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma. Single domain or single chain variable fragments lack the constant Fe region found in complete antibody molecules, and thus, the common binding sites (e.g., protein A/C) used to purify antibodies (single chain antibodies include the Fe region). These fragments can often be purified/immobilized using Protein L since Protein L interacts with the variable region of kappa light chains.

Flexible linkers generally are comprised of helix- and turn-promoting amino acid residues such as alaine, serine and glycine. However, other residues can function as well. Tang et al. (1996) used phage display as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the V_{H} C terminus and an abundance of arginines and prolines at other positions as the only common features of the selected tethers.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to mike fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

B. Pharmaceutical Formulations and Routes of Administration

The agents of the present disclosure may be administered by a variety of methods, e.g., orally or by injection (e.g. subcutaneous, intravenous, intraperitoneal, etc.). Depending on the route of administration, the active agent may be coated in a material to protect the agent from the action of acids and other natural conditions which may inactivate the agent.

The agents of the present disclosure may also be formulated and/or prepared in a variety of ways, including as a solid dispersion. See, for example, PCT Publication WO 2010/093944, which is incorporated herein by reference in its entirety.

To administer the therapeutic agent by other than parenteral administration, it may be necessary to coat the agent with, or co-administer the agent with, a material to prevent its inactivation. For example, the therapeutic agent may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al, 1984).

The therapeutic agent may also be administered systemically. Liquid or semi-liquid dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (i.e., the therapeutic compound) plus any additional desired ingredient from a previously sterile-filtered solution thereof.

The therapeutic agent can be orally administered, for example, with an inert diluent or an assimilable edible carrier. The therapeutic compound and other ingredients may also be enclosed in a hard or soft shell gelatin capsule, compressed into tablets, or incorporated directly into the subject's diet. In some embodiments, the agent is formulated as a capsule. For oral therapeutic administration, the therapeutic compound may be incorporated with excipients and used in the form of ingestible tablets, buccal tablets, troches, capsules, elixirs, suspensions, syrups, wafers, and the like. In some embodiments, the agent is formulated as an ingestible tablet. The percentage of the therapeutic agent in the compositions and preparations may, of course, be varied. The amount of the therapeutic agent in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specification fair the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient.

Active agents are administered at a therapeutically effective dosage sufficient to treat a condition associated with a condition in a patient. For example, the efficacy of a agent can be evaluated in an animal model system that may be predictive of efficacy in treating the disease in humans, such as the model systems shown in the examples and drawings.

The actual dosage amount of a compound of the present disclosure or composition comprising, an agent of the present disclosure administered to a subject may be determined by physical and physiological factors such as age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In certain embodiments, a pharmaceutical composition of the present disclosure may comprise, for example, at least about 0.1% of a compound of the present disclosure. In other embodiments, the compound of the present disclosure may comprise between about 2% to about 75% of the weight of the unit, or between about 25% to about 60%, for example, and any range derivable therein.

Single or multiple doses of the agents are contemplated. Desired time intervals for delivery of multiple doses can be determined by one of ordinary skill in the art employing no more than routine experimentation. As an example, subjects may be administered two doses daily at approximately 12 hour intervals. In some embodiments, the agent is administered once a day.

The agent(s) may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks therebetween. Alternatively, the predetermined routine schedule may involve administration on a twice daily basis for the first week, followed by a daily basis for several months, etc. in other embodiments, the invention provides that the agent(s) may taken orally and that the timing of which is or is not dependent upon food intake. Thus, for example, the agent can be taken every morning and/or every evening, regardless of when the subject has eaten or will eat.

The agents of the present invention may advantageously be incorporated into a comestible food directly ingestible by a user, i.e., foodstuffs, such as nutrient supplements, health drinks and probiotic foods. Generally, the components of the various types of food formulations will be conventional, although precise amounts of individual components and the presence of some of the conventional components may well be unconventional in a given formulation.

The food product may be a cooked product. It may incorporate meat or animal-derived material (such as beef, chicken, turkey, lamb, fish, blood plasma, marrowbone, etc or one or more thereof). The product alternative may be meat-free (preferably including a meat substitute such as soya, maize gluten or a soya product) in order to provide a protein source. The product may contain additional protein sources such as soya protein concentrate, milk, protein, gluten, etc. The product may also contain a starch source such as one or more grains (e.g., wheat, corn, rice, oats, barley, etc) or may be starch-free. The product may incorporate or be a gelatinized starch matrix. The product may incorporate one or more types of fiber such as sugar beet pulp, chicory pulp, chicory, coconut endosperm fiber, wheat fiber, etc. Dairy products may be suitable.

For many foods, it is accepted practice for the user to add the required amount of eggs in the course of preparation and this practice may be followed just as well herein. If desired, however, the inclusion of egg solids, in particular, egg albumen and dried yolk, in the food are allowable alternatives. Soy isolates may also be used herein in place of the egg albumen.

Dry or liquid flavoring agents may be added to the formulation. These include cocoa, vanilla, chocolate, coconut, peppermint, pineapple, cherry, nuts, spices, salts, flavor enhancers, among others. Acidulants commonly added to foods include lactic acid, citric acid, tartaric acid, malic acid, acetic acid, phosphoric acid, and hydrochloric acid. Other additives may include anti-oxidants, pH buffers, flavor masking agents, odor masking agents, preservatives, timed-release mechanisms, vitamins, minerals, electrolytes, hormones, herbal material, botanicals, amino acids, carbohydrates, fats, or the like.

### IV. COMBINATION THERAPY

In addition to being used as a monotherapy, the agents of the present invention may also find use in combination therapies. Effective combination therapy may be achieved with a single composition or pharmacological formulation that includes both agents, or with two distinct compositions or formulations, administered at the same time, wherein one composition includes a agents of this invention, and the other includes the second agent(s). Alternatively, the therapy may precede or follow the other agent treatment by intervals ranging from minutes to months.

Various combinations may be employed, such as when a compound of the present invention is “A” and “B” represents a secondary agent, non-limiting examples of which are described below:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

It is contemplated that other anti-inflammatory agents may be used in conjunction with the treatments of the current invention. For example, other COX inhibitors may be used, including arylcarboxylic acids (salicylic acid, acetylsalicylic acid, diflunisal, choline magnesium trisalicylate, salicylate, benorylate, flufenamic acid, mefenamic acid, meclofenamic acid and triflumic acid), arylalkanoic acids (diclofenac, fenclofenac, alclofenac, fentiazac, ibuprofen, flurbiprofen, ketoprofen, naproxen, fenoprofen, fenbufen, suprofen, indoprofen, tiaprofenic acid, benoxaprofen, pirprofen, tolmetin, zomepirac, clopinac, indomethacin and sulindac) and enolic acids (phenylbutazone, oxyphenbutazone, azapropazone, feprazone, piroxicam, and isoxicam. See also U.S. Pat. No. 6,025,395, which is incorporated herein by reference.

Other dietary agents may be combined, as are well known in the art. In addition, a dietary restriction such as low fat and/or low calorie diets may constitute a “combination” treatment with agents of the present invention.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Methods

Mice.

WT C5713116 mice were obtained from Jackson Laboratories, Harlan Laboratories, or the National Cancer Institute (NCI). LTα^{-/-}, LTβR^{-/-} and RORγt^{-/-} mice were bred at the University of Chicago. In cases of all heterozygous animals, breedings were set up where one parent was a knock-out and the other was a heterozygous animal. Mice were genotyped by PCR and weaned as early as 21 days and as late as 28 days after birth. Germ Free C57BL/6 mice were maintained in the Gnotobiotic facility at the University of Chicago. Mice were maintained according to the standards set by the University of Chicago's IACUC (Protocol #71866 and #58771).

IUD and NCD Challenge Experiments.

All SPF mice were maintained on Harlan Teklad 2918 until the start of diet where they were either switched onto 88137 or maintained on 2918 for the duration of the experiment. Mice were weighed every 7-10 days after the start of diet. At the end of diet (63-70 days after the start of diet), mice were sacrificed by CO₂ euthanasia and cervical dislocation. Mice were weighed again after sacrifice and perigonadal fat (periuterine or epididymal fat in the case of female and male mice respectively) was dissected and weighed.

Food Consumption.

Mice were started on either NCD or HFD at day zero and food was measured daily. Successive weights were subtracted from the previous day measured and data is plotted adjusted for days between measurements (1-3). 5 mice were housed in a cage.

Cecal and Stool DNA Extraction.

Cecal samples were collected at the time of sacrifice (at the end of NCI) or HFD respectively) and frozen at −80° C. until time of processing. Stool samples were collected freshly at 0, 4, and 9 weeks after the start of diet and frozen at −20° C. All extraction was done utilizing the QIAamp® DNA Stool Mini Kit (Qiagen, Alameda, Calif.) from Qiagen. Briefly, samples were lysed in a detergent solution and mechanically dissociated using a Mini-beadbeater from Biospec products (BioSpec Products, Bartlesville, Okla.) for 90 seconds at maximum setting. Samples were treated with InhibitEX matrix to prevent DNA damage and to inhibit PCR disrupting agents. Subsequently, proteins were digested with Proteinase K, samples were bound to a column, washed twice, and eluted in the supplied buffer. Quality of DNA and concentrations were determined utilizing Nanodrop.

PCR Amplification and 454 Pyrosequencing of 16S rDNA.

Sequencing and analysis were done as described in Proyko, 2010. V1-V2 regions of 16S rDNA from stool or cecal samples were amplified with TaKaRa Ex Taq PCR mixture (TAKARA Bio USA, Madison, Wis.). The PCR program was set at 95° C. 10 min, 30 cycles of 95° C. 1 min, 50° C. 1 min, 72° C. 1.5 min, followed by 72° C. for 10 minutes. PCR products were purified using the AMPure Kit (Agentcourt Bioscience, Beverly, Mass.). The resulting product was analyzed on a 2% agarose gel and by nanodrop. Products were then pooled at equal concentrations and sequenced on a GS Titanium 70×75 picotitre plate according to the manufactuerer's protocols for GS FLX (Roche Applied Science, Indianapolis, Ind.) at the Roy J Carver Center at the University of at Urbana Champaign.

Analysis of Pyrosequeneing.

Sequences were sorted analyzed using Mothur (Schloss, 2009). Each sample had between 2830-6043 sequences for an estimated depth of coverage greater than 89% for all samples. 16S rDNA sequence analysis was performed via MOTFIUR programs, version I.17.0. Low quality sequences were trimmed, redundant sequences were removed, chimeric sequences were removed by the Chimera slayer command, and sequences were aligned to the SILVA reference database. Principle Coordinate Analysis was addressed using the Yue & Clayton measure of dissimilarity. Samples were classified and differential abundance was detected for phyla level analysis using Metastats (White, 2009).

Sequence Alignment.

OTU alignment to the known 16s SFB rRNA encoding V1-V2 region was performed utilizing ClustalW2 available from the European Bioinformatics Institute.

Germ Free Experiments.

Germ free NCD and HFD were described in Table 1. WT C57BL/6 germ free mice were savaged with fresh caecal contents from LTβR^{+/-} and LTβR^{-/-} donors maintained on similar diets.

Colon Culture and ELISA.

Proximal colon pieces weighing less than 0.05 g were cut in small pieces and incubated in 0.4 mL of RPMI 1640x containing 10% FBS, amphotericin, gentamicin, penicillin, and streptomycin for 48 hr in tissue plates, as previously described by Zheng et al. (Zheng, 2008) IL-23 in supernatants was measured by ELISA (eBiosciences) according to the manufacturer's recommendations.

Hydrodynamic Injection.

Hydrodynamic injection was performed as described by Tumanov et by placing mice in a conical restraining device with an attached heating element. 10 μg of a plasmid vector expressing IL-22 (IL22, Genentech) or empty vector (pERK) were injected two days prior to the start of HFD in 1.8 mL TransIT-EE Hydrodynamic Delivery Solution (MIR 5340, Mirus Bio LLC) over a period that lasted less than five seconds.¹⁶

Real-Time PCR.

RNA was extracted from colon samples frozen at −80° C. in RNALater. Briefly, samples were homogenized in TRizol Reagent® (Invitrogen, Carlsbad, Calif.) and underwent phenol-chloroform extraction. The product was treated with Amplification Grade DNAse I available from Sigma Aldrich (Sigma Aldrich Corporation, St Louis, Mo.). Product integrity was verified by running samples on 2% agarose gels. 2 μg of RNA was utilized to make cDNA using M-MUTV Reverse Transcriptase and associated buffers, dNTPs, and oligo-dT primer from Promega (Promega, Fitchburg, Wis.). Samples were amplified on an ABI 7900 instrument (Applied Biosystems Inc, Foster City, Calif.) using SsoFast™ EvaGreen® Supeimix (Bio-Rad Laboratories, Hercules, Calif.); primer concentrations were 0.5 μM in the final reaction. Correct melting temperatures for all products were verified after amplification. For all products, amplification in all samples resulted in correct melting temperatures. For IL-22 and RegIIIβ targets, amplification often resulted in multiple products and reactions with the resulted in multiple products are excluded from both groups. For IL-22, no LTβR^{-/-} animal produced a product with the correct melting temperature, likely due to the low transcript level for this product in these mice. Amplification data for all PCR reactions was submitted to Real-Time PCR Miner for accurate Ct value calculation and Primer Efficiency assessment (Zhao, 2005). Fold relative to WT normalized to HPRT was calculated utilizing the PfaffI method. Primers were as follows:

| | | | SEQ |
|---|---|---|---|
| | | | ID |
| Primer | Sequence | Reference | No. |
| HPRT | HPRTF: TGAAGAGCTACTGTAAT | Tumanov | 1 |
| | GATCAGTCAAC | | |
| | HPRTR: AGCAAGCTTGCAACCTT | Tumanov | 2 |
| | AACCA | | |
| IL23p19 | IL23p19f: GGT GGC TCA GGG | Zheng | 3 |
| | AAA TGT | | |
| | IL23p19R: GAC AGA GCA GGC | Zheng | 4 |
| | AGG TAC AG | | |
| TGFβ | TGFβF: CACTGATACGCCTGAGTG | Firan | 5 |
| | TGFβR: GTGAGCGCTGAATCGAAA | Firan | 6 |
| IL-6 | IL6F: TCC AAT GCT CTC CTA | Zheng | 7 |
| | ACA GAT AAG | | |
| | 1L6R: CAA GAT GAA TTG GAT | Zheng | 8 |
| | GGT CTT G | | |
| 1L-17A | IL-17A Ex2F ctccagaaggccc | Ivanov | 9 |
| | tcagactac | | |
| | IL-17A Ex3R agctttccctccg | Ivanov | 10 |
| | cattgacacag | | |
| IL-17F | IL-17F Ex1F gaggataacactg | Ivanov | 11 |
| | tgagagttgac | | |
| | IL-17F Ex2R2 gagttcatggtg | Ivanov | 12 |
| | ctgtcttcc | | |
| IL-22 | 1L22F: TCC GAG GAG TCA GTG | Zheng | 13 |
| | CTA AA | | |
| | IL22R: AGA ACG TCT TCC AGG | Zheng | 14 |
| | GTG AA | | |
| RegIIIγ | RegIIIgF: ATG GCT CCT ATT | Zheng | 15 |
| | GCT ATG CC | | |
| | RegIIIgR: GAT GTC CTG AGG | Zheng | 16 |
| | GCC TCT T-3′ | | |
| RegIIIβ | RegIIIbF: ATG GCT CCT ACT | Zheng | 17 |
| | GCT ATG CC | | |
| | RegIIIbR: GTG TCC TCC AGG | Zheng | 18 |
| | CCT CTT T | | |
| EUA | EUAF: ACTCCTACGGGAGGCAGCA | Barman | 19 |
| | GT | | |
| | EUAR: ATTACCGCGGCTGCTGGC | Barman | 20 |
| SFB | SFBF: GACGCTGAGGCATGAGAGC | Barman | 21 |
| | AT | | |
| | SFBR: GACGGCACGGATTGTTATT | Barman | 22 |
| | CA | | |

Sequencing Primers/Adaptors were:

| | | SEQ |
|---|---|---|
| | | ID |
| | | NO. |
| TA-27FMID1 | CGTATCGCCTCCCTCGCGCCATCAGACGAGTGCGTAGAGTTTGATCCTGGCTCAG | 23 |
| TA-27FMID2 | CGTATCGCCTCCCTCGCGCCATCAGACGCTCGACAAGAGTTTGATCCTGGCTCAG | 24 |
| TA-27FMID3 | CGTATCGCCTCCCTCGCGCCATCAGAGACGCACTCAGAGTTTGATCCTGGCTCAG | 25 |
| TA-27FMID4 | CGTATCGCCTCCCTCGCGCCATCAGAGCACTGTAGAGAGTTTGATCCTGGCTCAG | 26 |
| TA-27FMID5 | CGTATCGCCTCCCTCGCGCCATCAGATCAGACACGAGAGTTTGATCCTGGCTCAG | 27 |
| TA-27FMID6 | CGTATCGCCTCCCTCGCGCCATCAGATATCGCGAGAGAGTTTGATCCTGGCTCAG | 28 |
| TA-27FMID7 | CGTATCGCCTCCCTCGCGCCATCAGCGTGTCTCTAAGAGTTTGATCCTGGCTCAG | 29 |
| TA-27FMID8 | CGTATCGCCTCCCTCGCGCCATCAGCTCGCGTGTCAGAGTTTGATCCTGGCTCAG | 30 |
| TA-27FMID9 | CGTATCGCCTCCCTCGCGCCATCAGTAGTATCAGCAGAGTTTGATCCTGGCTCAG | 31 |
| TA-27FMID10 | CGTATCGCCTCCCTCGCGCCATCAGTCTCTATGCGAGAGTTTGATCCTGGCTCAG | 32 |
| TA-27FMID11 | CGTATCGCCTCCCTCGCGCCATCAGTGATACGTCTAGAGTTTGATCCTGGCTCAG | 33 |
| TA-27FMID13 | CGTATCGCCTCCCTCGCGCCATCAGCATAGTAGTGAGAGTTTGATCCTGGCTCAG | 34 |
| TA-27FMID14 | CGTATCGCCTCCCTCGCGCCATCAGCGAGAGATACAGAGTTTGATCCTGGCTCAG | 35 |
| TB-338R | CTATGCGCCTTGCCAGCCCGCTCAGTGCTGCCTCCCGTAGGAGT | 36 |

### Example 2

### Results

LTβR and LTα are Essential for Weight Gain in DIO.

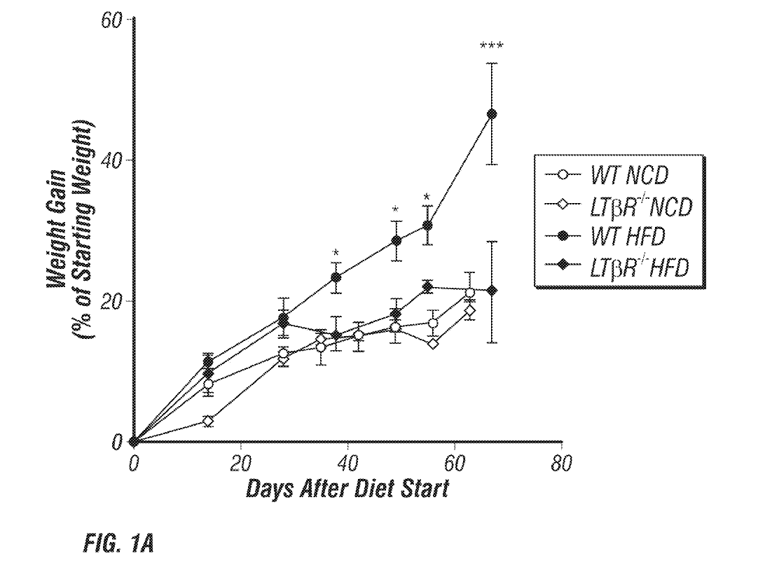

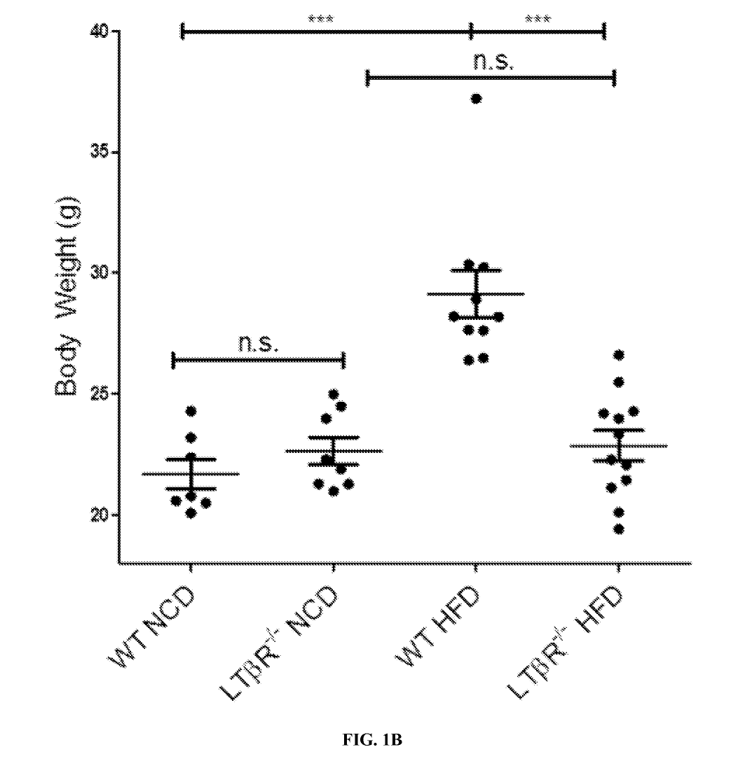

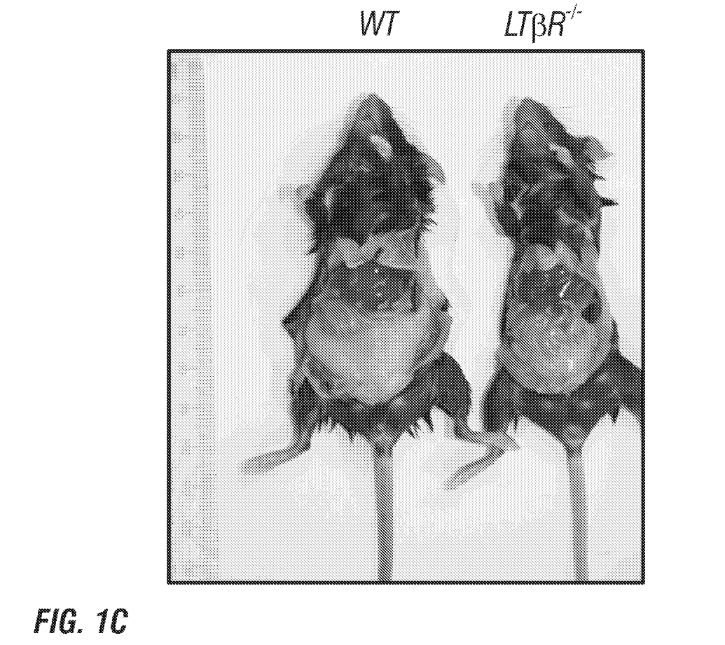

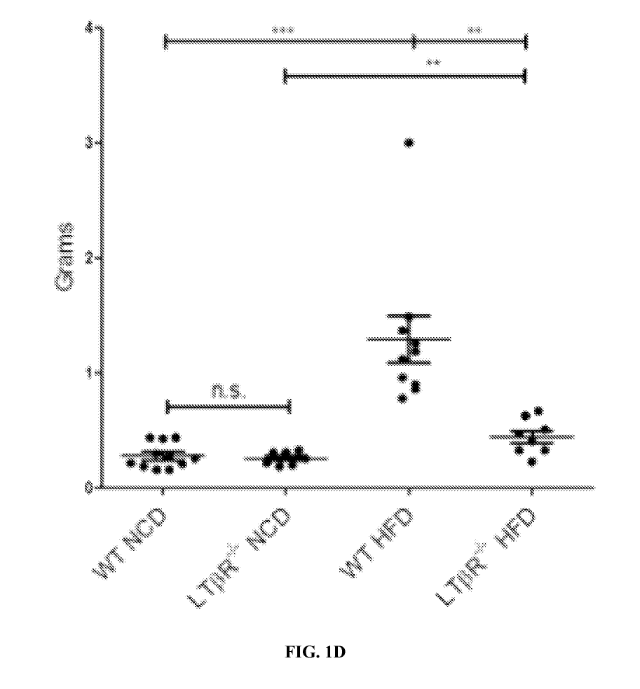

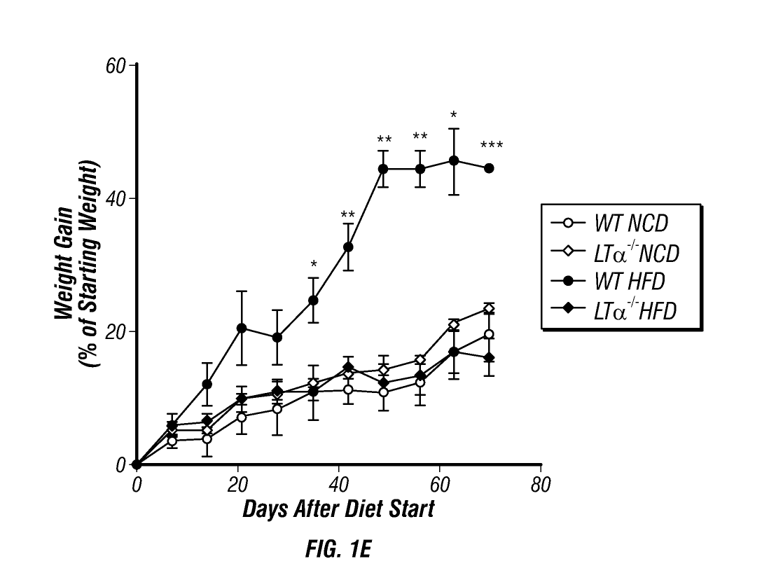

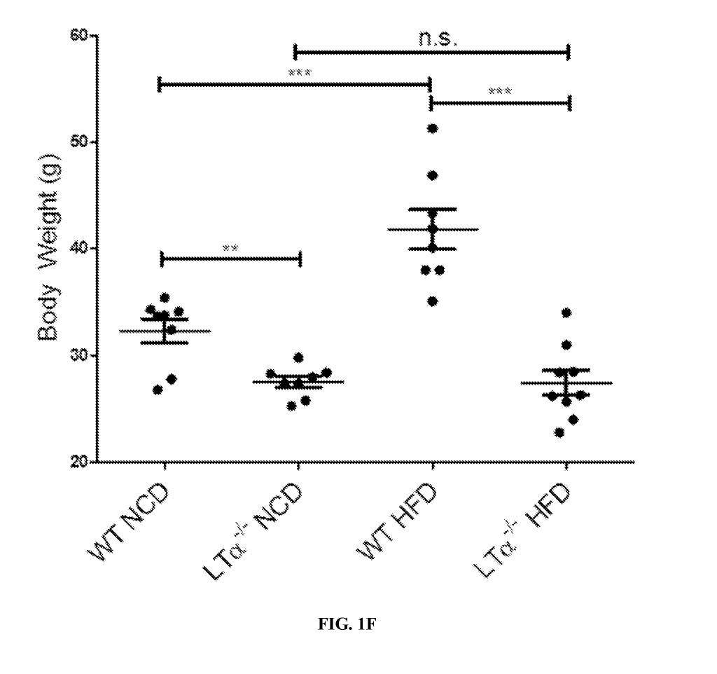

In order to address the role of the LT pathway in DIO, the inventor challenged WT and LTβR^{-/-} adult animals with HFD. Animals were kept on normal chow diet (NCD) until 9 weeks of age where they were either switched onto HFD or maintained on NCD (for composition of all diets see Table 1). While there was no difference in growth between WT and LTβR^{-/-} mice on NCD, WT mice on HFD gained significantly more weight than LTβR^{-/-} animals, which were resistant to DIO (FIG. 1A). There was no difference in weight after 9 weeks of dietary challenge between WT and LTβR^{-/-} animals maintained on NCD; WT and LTβR^{-/-} animals weighed 21.70±0.60 g and 22.66±0.56 g at the end of NCI) respectively (FIG. 1B). However, at the end of HFD, WT and LTβR^{-/-} groups were significantly different, weighing 29.13±0.99 g and 22.87±0.62 g respectively (FIG. 1B). In contrast to WT mice, LTβR^{-/-} animals do not gain additional weight after prolonged HFD, suggesting a role for the LT pathway in controlling excess weight gain induced by HFD.

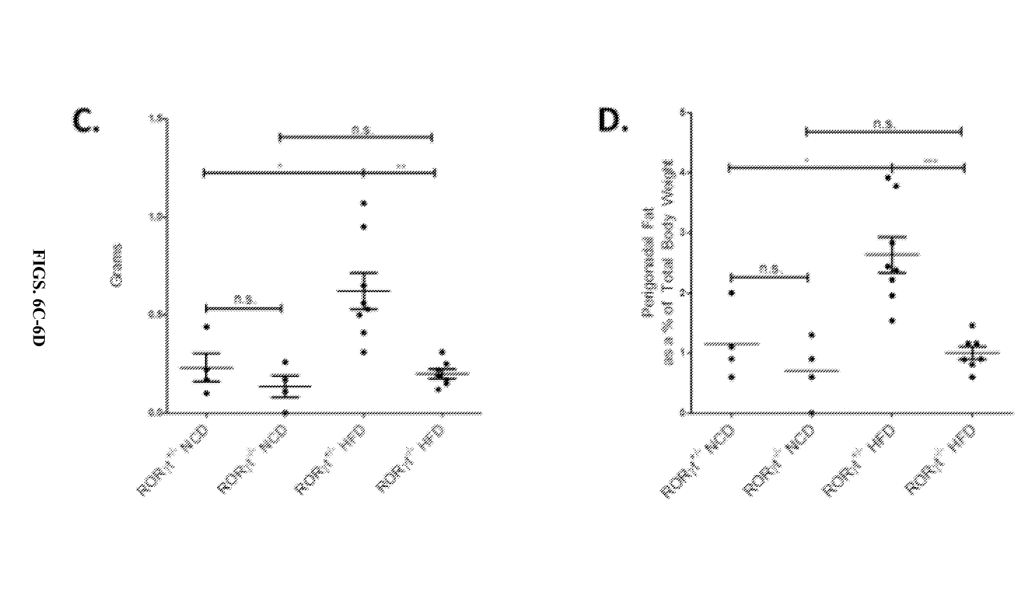

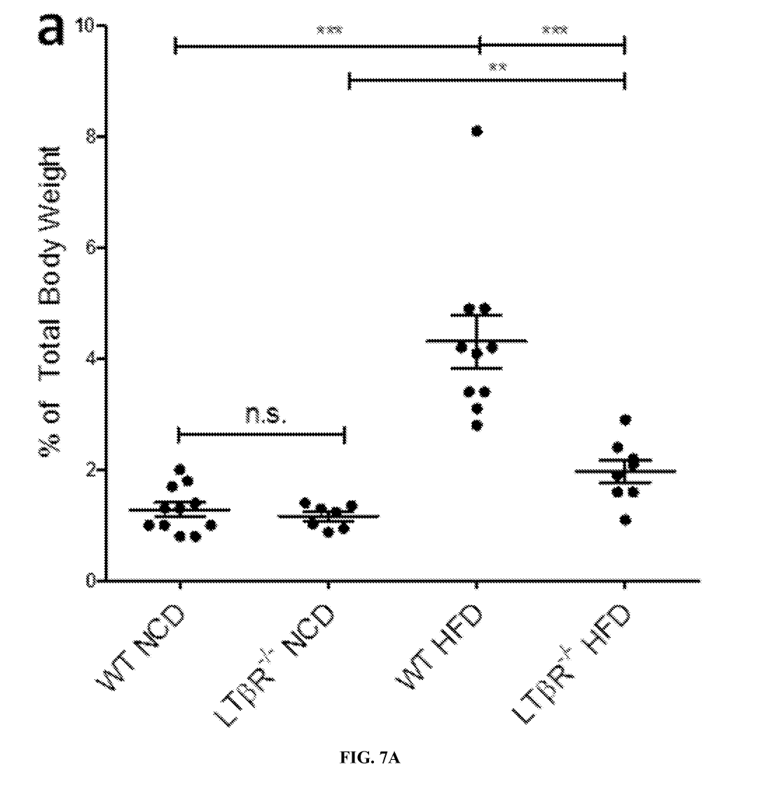

At the time of sacrifice it was clear that changes in weight gain corresponded with changes in adiposity. The perigonadal fat pad of WT animals was much larger than that of LTβR^{-/-} mice (FIG. 1C). To quantify these results, the perigonadal fat pad of WT and LTβR^{-/-} mice at the end of diet was dissected out and weighed. Both in absolute terms and as a percentage of body weight, the perigonadal fat pad of WT mice had expanded much more than that of LTβR^{-/-} mice on HFD. This is in stark contrast to their relative adiposity on NCD, where WT and LTβR^{-/-} animals weighed similarly at the end of diet and had similar body composition (FIG. 1D and FIG. 7). Together, this data demonstrates that LTβR is essential for excess weight gain and adiposity induced by HFD.

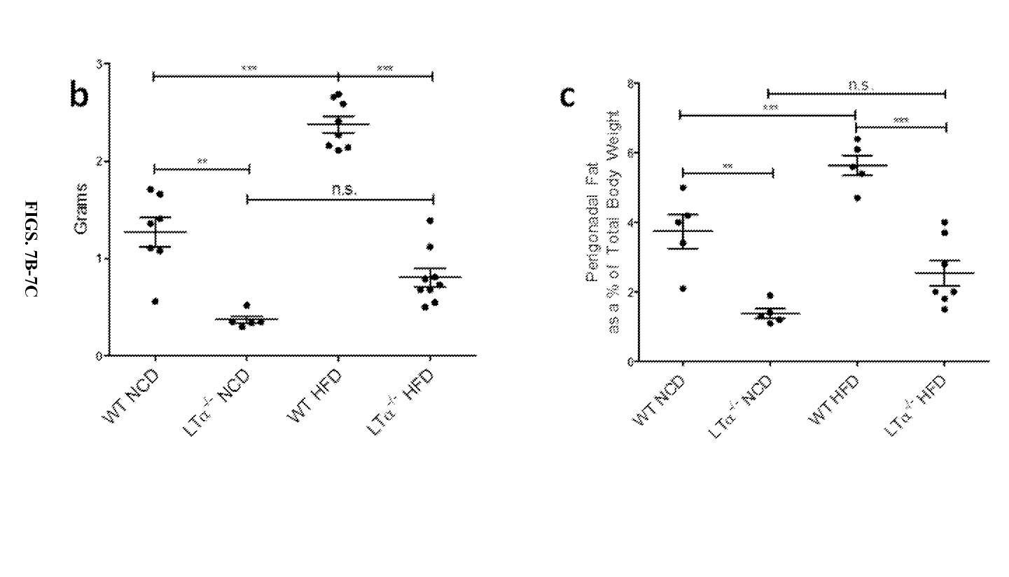

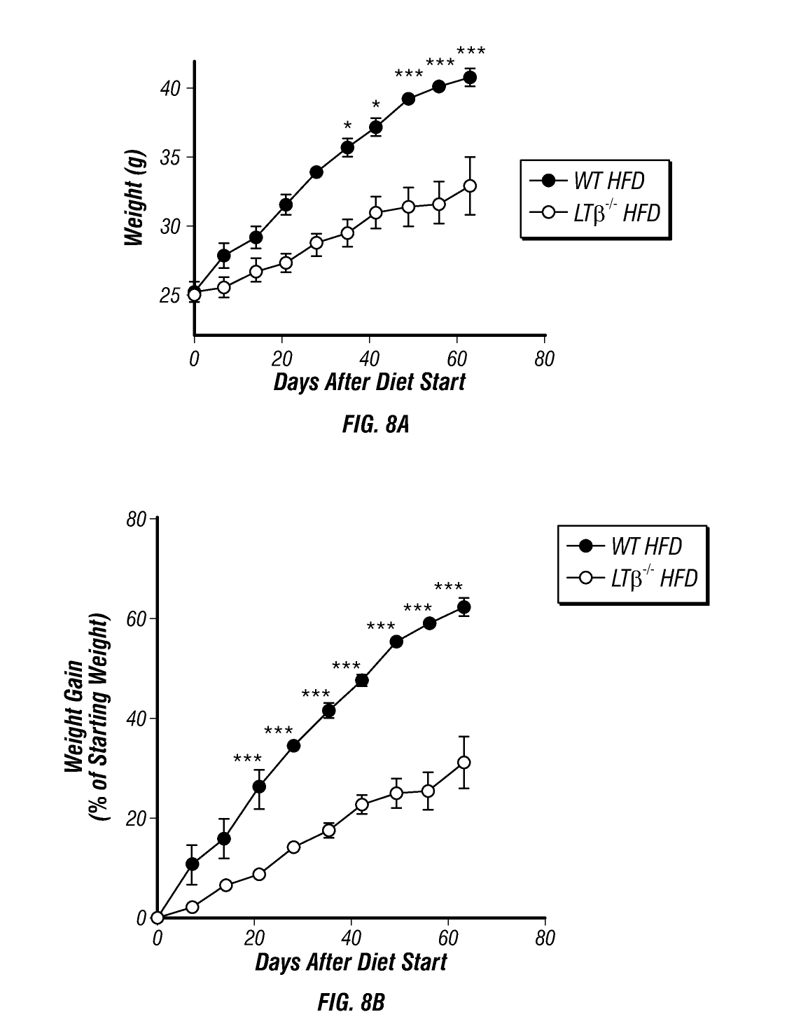

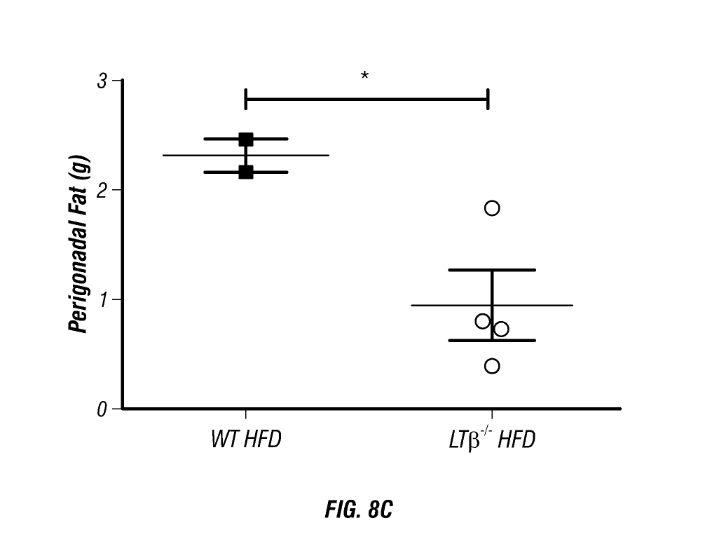

LTα forms part of a membrane bound heterotrimer that forms one of the key physiological ligands that bind to LTβR, and polymorphisms in coding exons of LTα have been linked to obesity¹³. The inventor therefore challenged WT and LTα^{-/-} mice with HFD to determine whether this ligand was essential for weight gain. Consistent with the results in LTβR^{-/-} animals, LTα^{-/-} animals resisted DIO and showed similar growth on HFD to both WT and LTα^{-/-} mice on NCD (FIG. 1D); these growth patterns reflected stark differences in body composition between WT and LTα^{-/-} animals on HFD, with the latter being much leaner than the former (FIG. 1E and FIG. 7). There was a modest, but detectable difference in weight between LTα^{-/-} and WT animals on NCD, and this could be attributed to additional agonism of the TNFR pathway (FIG. 1F). However, unlike WI animals and similar to LTβR mice, LTα−/− animals did not appear to increase body weight on HFD, contextualizing the significance of the LT pathway in DIO. Furthermore, LTβ−/− animals also resisted weight gain compared to WT animals on HFD (FIG. 8). Together, the data for LTα^{-/-}, LTβ−/− and LTβR^{-/-} animals demonstrates the importance of the intact membrane bound UT pathway in DIO.

LTβR Regulates Changes to the Microbiota that are Causative of Differential Weight Gain.

In order to better understand the mechanism by which the LT pathway promoted weight gain in DIO, the inventor addressed the food intake of WT and LTβR^{-/-} animals on NCD and HFD. There were no obvious changes in feeding behavior between both groups on NCD or on HFD (FIG. 2A), suggesting that differences in weight gain were occurring despite similar consumption patterns. Studies in germ free mice have revealed that the intestinal microbiota enable access to greater caloric intake, and as a result germ free mice weigh substantially less than their conventionalized littermates⁴. Because consumption patterns were similar between WT and LT-deficient animals and the LT-signaling pathways plays such a prominent role in normal mucosal defense, the inventor wondered if the LT-pathway influenced changes in the microbiota that promoted weight gain.

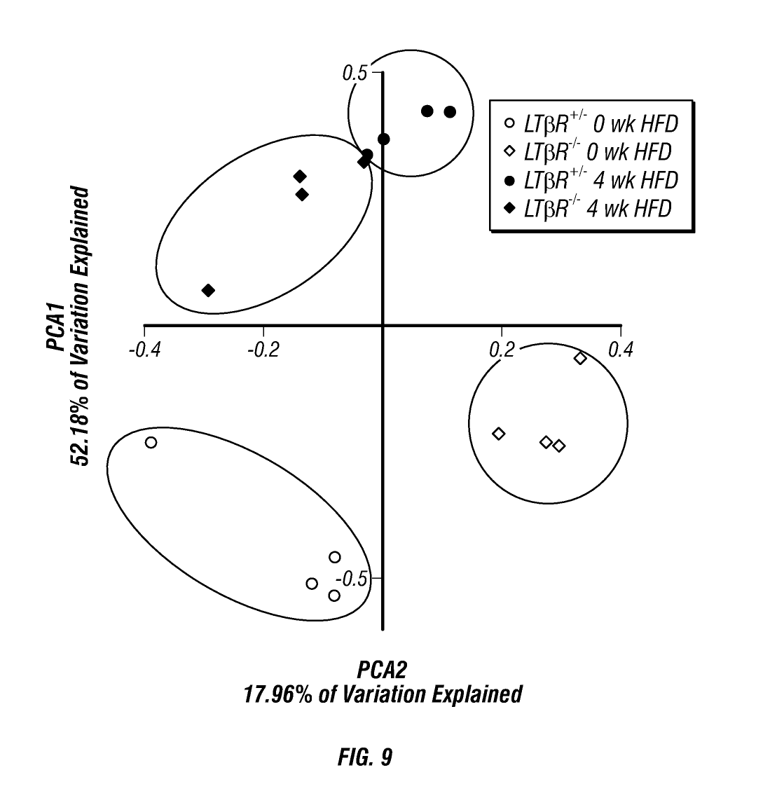

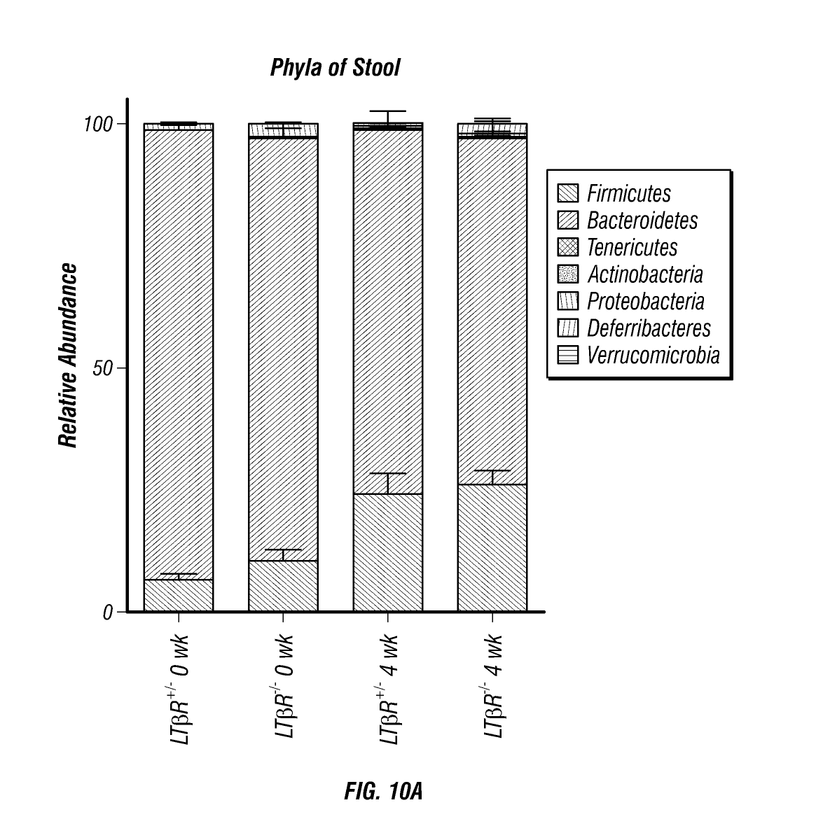

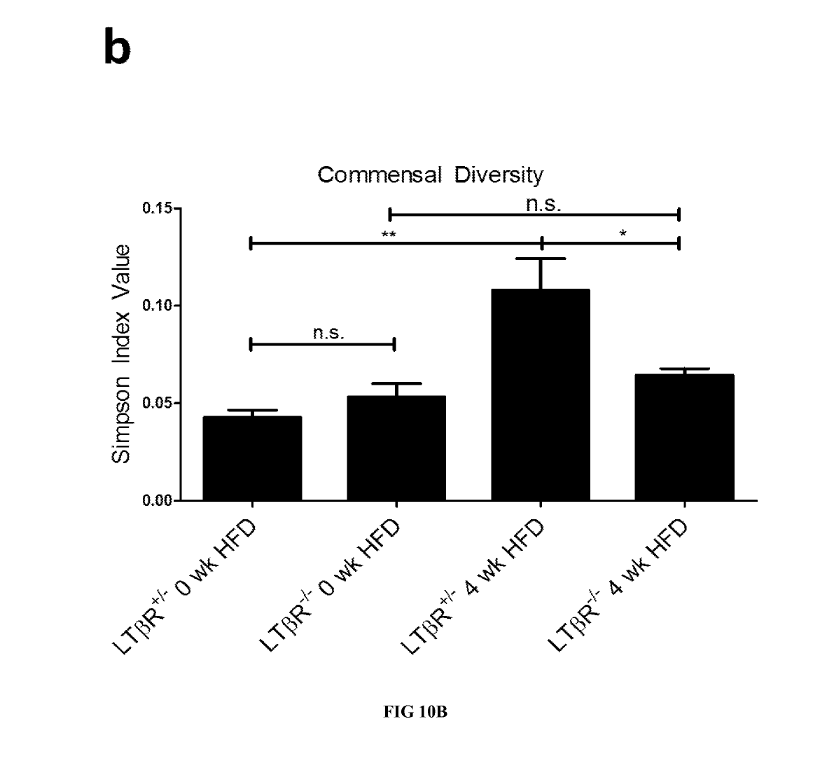

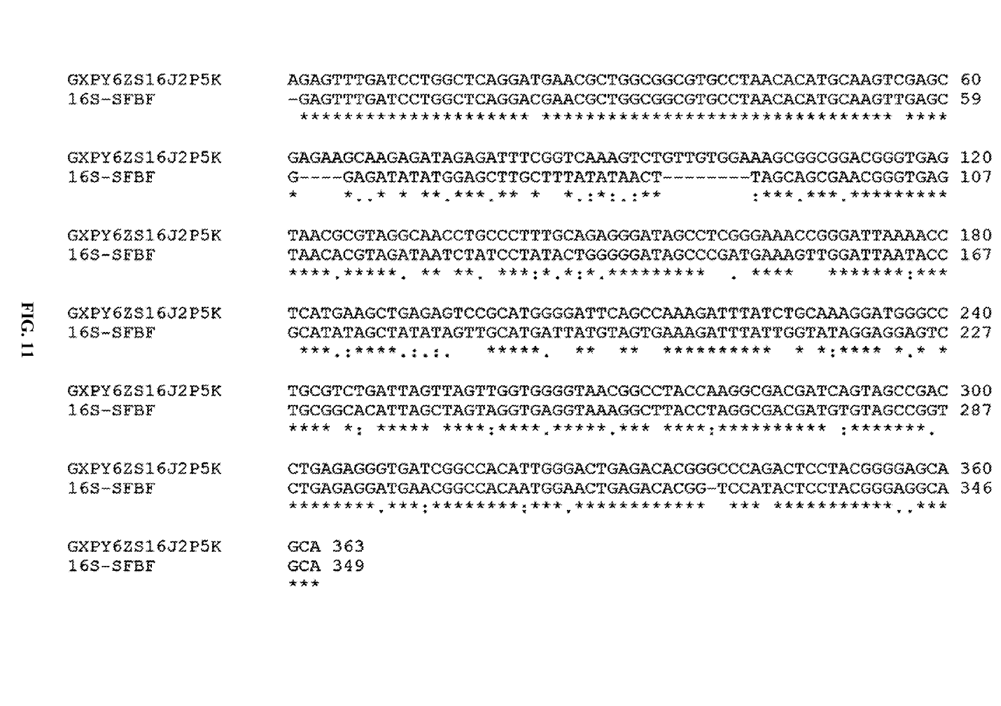

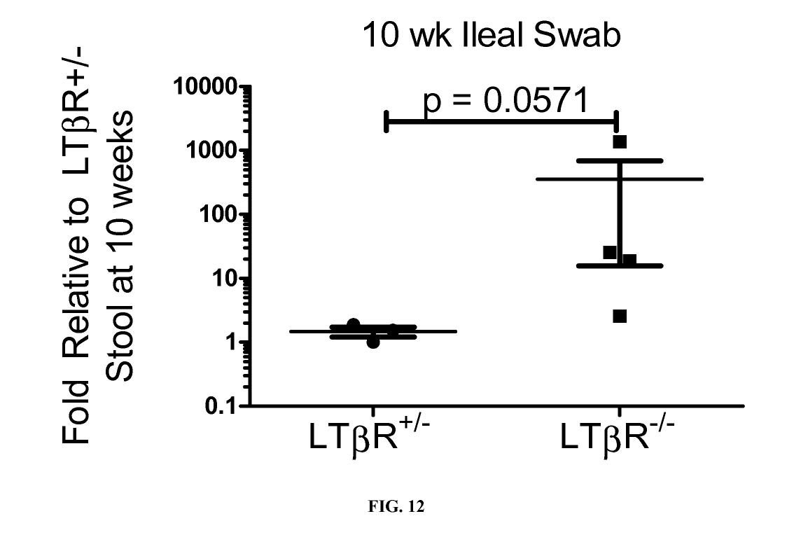

In order to address this issue, the inventor amplified the V1-V2 tags of 16s rRNA encoding genes from stool samples obtained from LTβR^{+/-} and LTβR^{-/-} animals on NCD and HFD and subjected the resulting PCR products to 454 Pyrosequencing. The inventor performed Principle Coordinate Analysis (PCA) to spatially discriminate the V1-V2 tag sequences of 16s rRNA encoding genes from LTβR⁺⁻ and LTβR^{-/-} stool DNA. PCA revealed genotype and diet specific clustering dependent on the two largest components of variation (FIG. 9). Intriguingly, PCA1 (52.18% of variation) strongly separated NCD and HFD groups and is consistent with a HFD-induced expansion of the Firmicute phyla observed in both groups (FIG. 10); PCA2 (17.96% of variation) separated knock-out and heterozygous animals, and demonstrated differences not explained by diet alone. In addition to Firmicute expansion, a hallmark of the “obese microbiome” in human stool is a loss of commensal diversity. The inventor was excited to note that LTβR+/− animals experienced reduced commensal diversity after the start of FEED, but that LTβR−/− animals maintained a similarly diverse community to either group at the start of diet (FIG. 10).

To test how the changes to the microbiota contributed to weight gain, the inventor transplanted the cecal contents of LTβR and LTβR mice into WT germ free recipients. Recipients were maintained on a diet of similar composition to their donors (Table 1). Consistent with the results in SPF mice, there was no difference in weight gain between recipients that received cecal contents from LTβR^{+/-} or LTβR^{-/-} donors on NCD after 20 days of diet (FIG. 2b), suggesting that although there were detectable differences in the microbial communitBes at this point, in and of themselves, these differences seen on NCD were unable to explain differential weight gain between genotypes. In contrast, the cecal contents of LTβR^{+/-} animals conferred greater weight gain than that of LTβR^{-/-} animals when both donor and recipient groups were kept on HFD (FIG. 2c). Although recipient groups weighed differently at and prior to 20 days after transplant, LTβR^{-/-} recipients caught up in weight gain after this time point (data not shown); eventual leveling out of growth could be due to the fact that the micro biota was donated into WT recipients and the intact immune response of these hosts influenced the microbiota as part of a normal regulatory circuit. This data demonstrates that changes in the microbial communities colonizing LTβR^{+/-} and LTβR^{-/-} animals after HFD are at least transiently causative of excess weight gain in the heterozygous group after HFD.

TABLE 1

| | Normal Chow | High Fat | Germ Free | Germ Free |
|---|---|---|---|---|
| Content | (NCD) | Diet (HFD) | NCD | HFD |
| Make/Catalog # | Harlan | Harlan | Harlan | Harlan |
| | Teklad 2918 | Teklad | Teklad | Teklad |
| | | 88137 | 2016S | 97222 |
| % kCal | 6.2% | 42.0% | 12% | 37.4% |
| Fat | | | | |
| % kCal | 58% | 42.7% | 66% | 46.8% |
| Carbohydrate | | | | |
| % kCal | 24% | 15.2% | 22% | 15.8% |
| Protein | | | | |
| Total Energy per | 3.1 kJ/g | 4.5 kJ/g | 3.0 kJ/g | 4.4 kJ/g |
| Gram | | | | |

The fecal stream is composed of allochthonous (transient) and autochthonous (permanent resident) microbes and is informative of microbiota living throughout the gastrointestinal tract. Analysis of stool revealed changes in specific operational taxonomic units (OTUs) between heterozygous and knockout animals 4 weeks after HFD. There were several OTUs overrepresented in LTβR^{-/-} mice after HFD, and a possible interpretation of such overrepresentation is that these are species whose clearance was dependent on LTβR after HFD. Such clearance could contribute to the loss of commensal diversity experienced by heterozygous animals after HFD was initiated (FIG. 10). One OTU significantly overrepresented in LTβR^{-/-} animals was not classifiable beyond the Clostridiales order (Table 2). The OTU detected in the inventor's analysis had high sequence homology with the V1-V2 region of the 16S rRNA encoding gene of Segmented Filamentous Bacteria (SFB) (FIG. 10), an autochthonous, unclassified Clostridiales order member that provokes a Th17 cytokine based immune response (Wu, 2010; Klaasen, 1993; Ivanov, 2009). SFB is detectable in the fecal stream of mice throughout adulthood (Sczesnak, 2011; Prakash, 2011). Quantitative PCR with primers specific for SFB demonstrated that SFB experienced a moderate overgrowth in LTβR^{-/-} animals but was greatly reduced in WT animals, especially in response to HFD (FIG. 2D and FIG. 11). Therefore, the LTβR pathway regulates changes, including loss of commensal diversity, after the initiation of HFD.

TABLE 2

| | | Numerical | | |
|---|---|---|---|---|
| | | Classification | | |
| p-value | | (Mothur ID) via | Interpretation of | Fold |
| Rank | p-value | SILVA Database | Classification | KO/Het |
| 1 | 0.0073 | 0.1.15.2.5 | Unclassified | 3.25 |
| | | | Clostridiales | |
| 2 | 0.0073 | 0.1.15.2.5.1 | Unclassified | 3.25 |
| | | | Clostridiales | |
| 3 | 0.0073 | 0.1.15.2.5.1.1 | Unclassified | 3.25 |
| | | | Clostridiales | |
| 4 | 0.0140 | 0.1.24.4.1.2.6 | Unclassified | 6.5 |
| | | | Helicobacteraceae | |
| 5 | 0.0320 | 0.1.34 | Unclassified Bacteria | 0.27 |
| 6 | 0.0320 | 0.1.34.1 | Unclassified Bacteria | 0.27 |
| 7 | 0.0320 | 0.1.34.1.1 | Unclassified Bacteria | 0.27 |
| 8 | 0.0320 | 0.1.34.1.1.1 | Unclassified Bacteria | 0.27 |
| 9 | 0.0320 | 0.1.34.1.1.1.1 | Unclassified Bacteria | 0.27 |

Weight Gain and SFB Regulation were Transmissible by Housing LTβR Deficient Mice with their Obesity Prone Siblings.

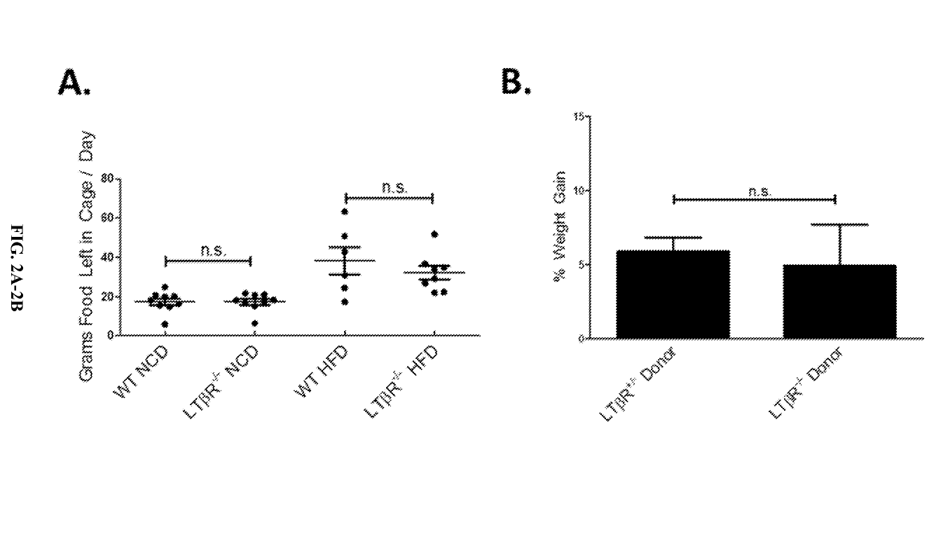

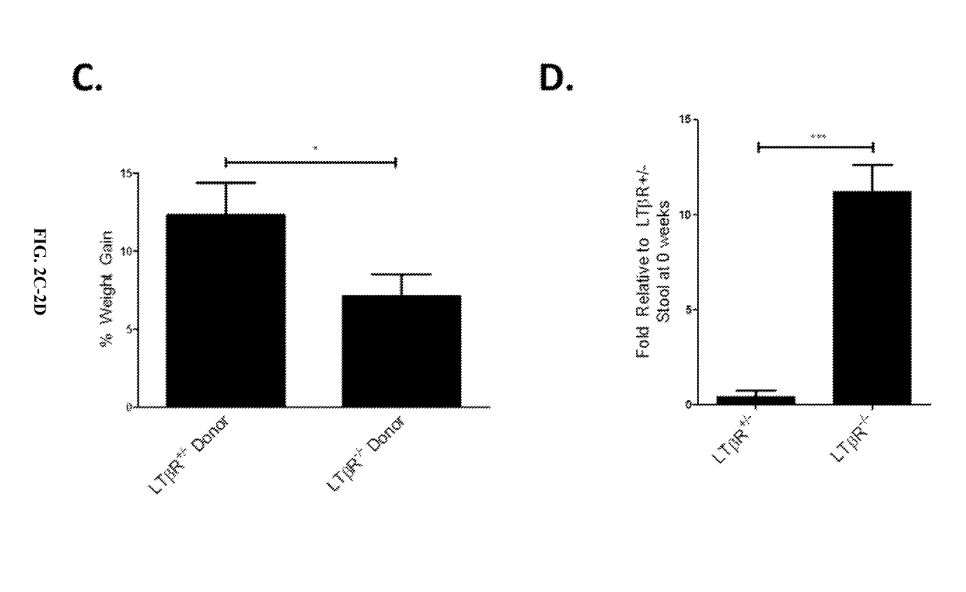

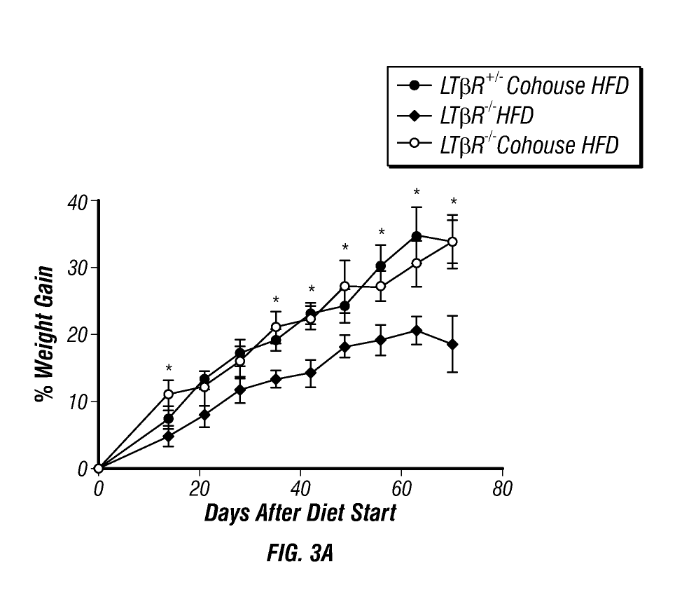

Given the conflicting viewpoints presented by various twin studies regarding genetic and environmental causes for obesity (Stunkard, 1986a; Stunkard, 1986b; Muegge, 2011), the inventor wondered whether environmental manipulation would influence the phenotype of LTβR deficient animals. In order to explore this, LTβR^{+/-} and LTβR^{-/-} littermates were weaned into cages separated by genotype or into cages where genotypes were mixed. Mice are coprophagic and fecal consumption is a mechanism by which mice housed in the same cage constantly colonize one another; cohousing is a commonly exploited experimental technique to facilitate microbiota exposure (Ivanov, 2009; Lathrop, 2011), While separately housed, LTβR^{-/-} mice resisted excess body weight deposition induced by diet, but cohousing LTβR^{-/-} mice with their LTβR^{+/-} littermates rescued excess weight gain in the LT-deficient group (FIGS. 3A-4). The data suggests that LTβR^{+/-} littermates, which maintain intact regulation of their own microbiota, maybe constantly exposing LTβR^{-/-} mice to their own obesity-inducing microbes and supplementing growth. Although both mice are exposed to the other's microbiota, it is clear that excess weight gain is the dominant phenotype in these experiments and correlates with rescued regulation of the microbiota.

Because species diversity loss is a hallmark of the obese microbiome in humans, and because SFB is a species which reduces in abundance after HFD through an LTβR-dependent mechanism, the inventor used SFB as a representative marker species for changes to the microbiota. SFB levels dramatically decreased after heterozygous animals were placed on HFD (FIG. 3C). However LTβR^{-/-} animals separately housed from their LTβR^{+/-} littermates saw very modest decreases in SFB after HFD and actually sustained an overgrowth, consistent with the results for differential abundance, the inventor observed in 16S rRNA profiling (FIG. 3C). It is exciting to note that housing LTβR^{-/-} animals with their LTβR^{+/-} littermates rescued clearance of SFB, which correlated with increased weight gain (FIG. 3C). This data demonstrates that exposing LTβR^{-/-} mice with their LTβR replete siblings not only rescues weight gain, but rescues changes in the microbiota normally induced by exposure to HFD. The transmissibility of the obese phenotype tracked with changes in the microbiota normally associated with the obese state.

The LT-Pathway Selectively Influences the Innate IL-23/IL-22 Axis of Th17 Cytokine Members.

The behavior of SFB prompted us to consider elements of the Th17 cytokine pathway that might be regulated by LTβR, because this particular immune response relies on SFB for induction (Wu, 2010; Ivanov, 2009). TGFβ, IL-6, IL-17A, and IL-17F transcript levels were similar between LTβR^{+/-} and LTβR^{-/-} groups after HID and between groups on NCD (FIGS. 4A-D and FIG. 13). However, transcripts for IL23p19 and IL-22, a key downstream cytokine regulated by IL-23, were reduced in LTβR^{-/-} mice (FIGS. 4E-4F). Furthermore, IL-23p19 was induced by HFD as compared to the NCD state (FIG. 13). Additionally, members of the RegIII antimicrobial peptide family, which are downstream of the IL-23/IL-22 pathway were also greatly reduced in LTβR^{-/-} animals after HFD (FIGS. 4G-H). The selective loss of transcript in the IL-23/IL-22 pathway and not the IL-17A/F pathway suggested preferential involvement of this innate signaling axis in regulating the microbiota and DIO.

IL-23 is Regulated by LTβR and Necessary for DIO.

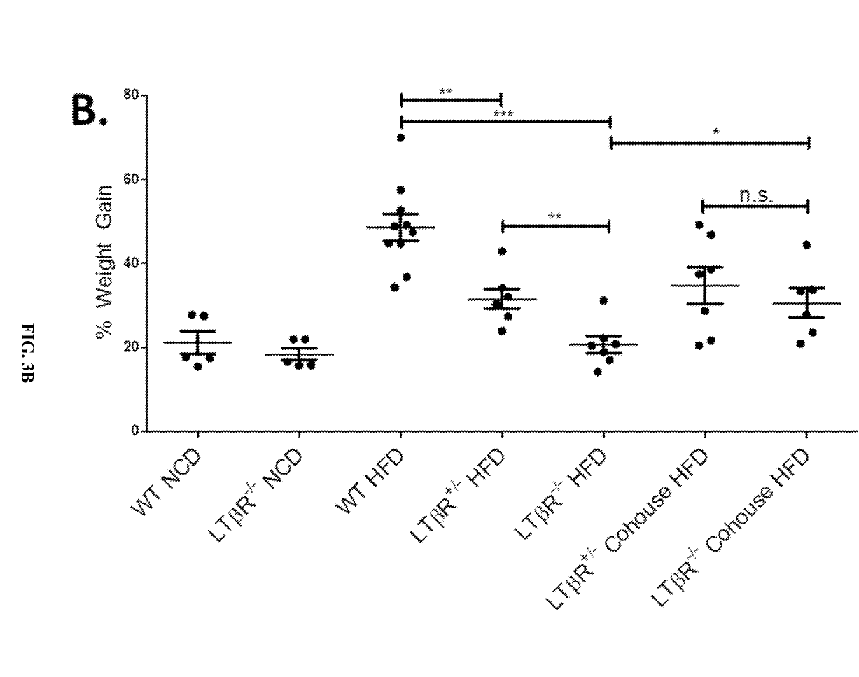

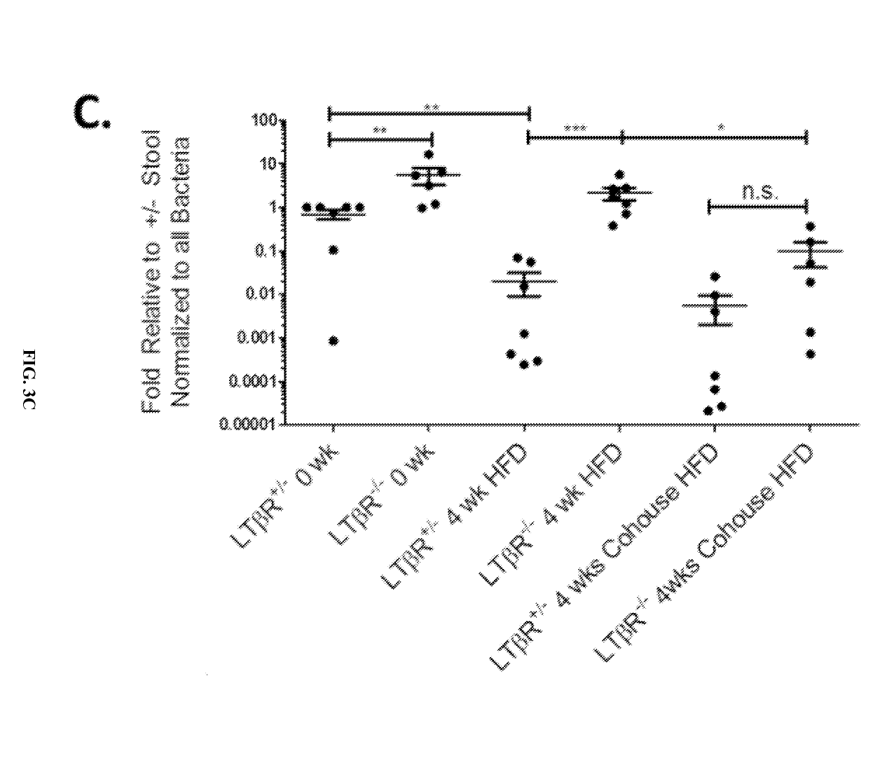

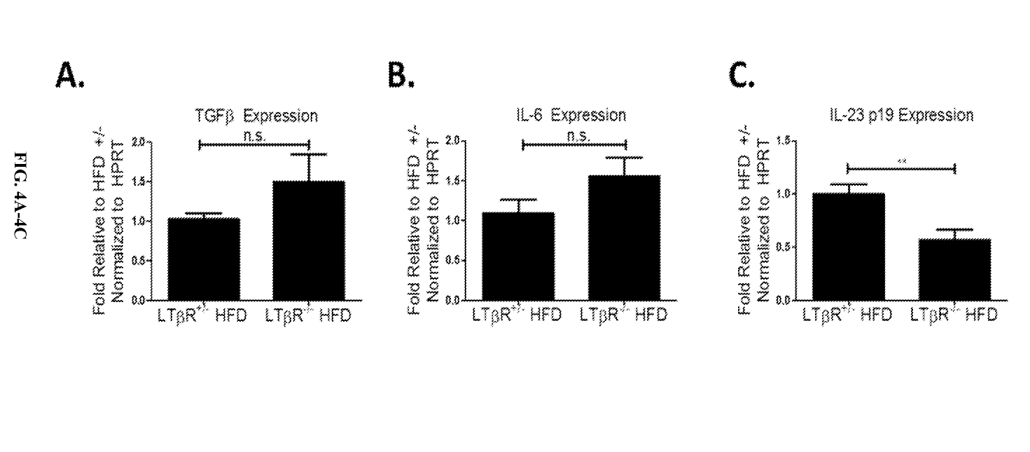

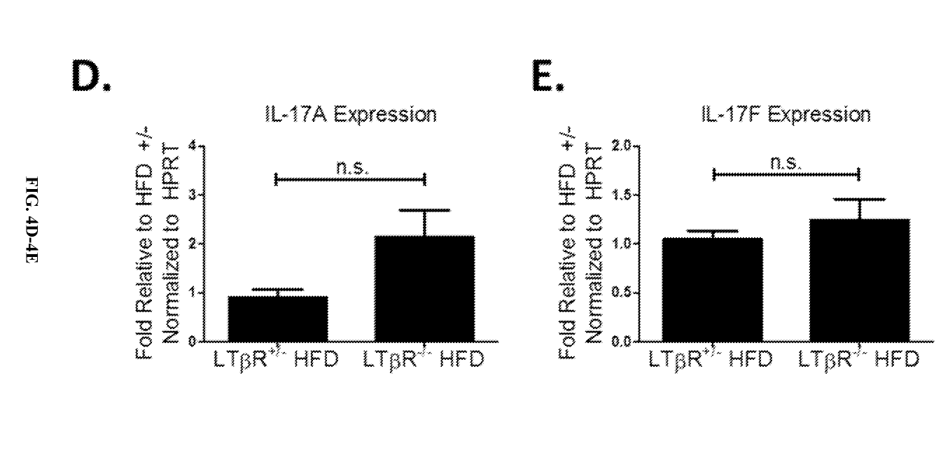

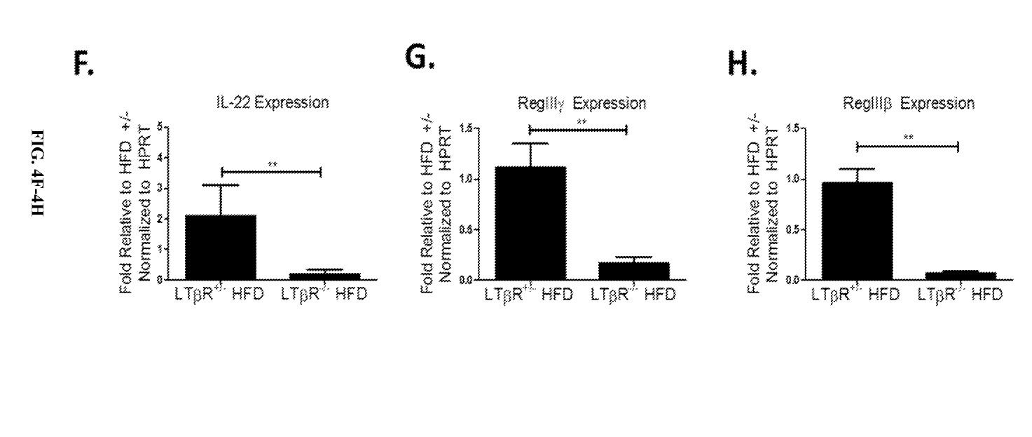

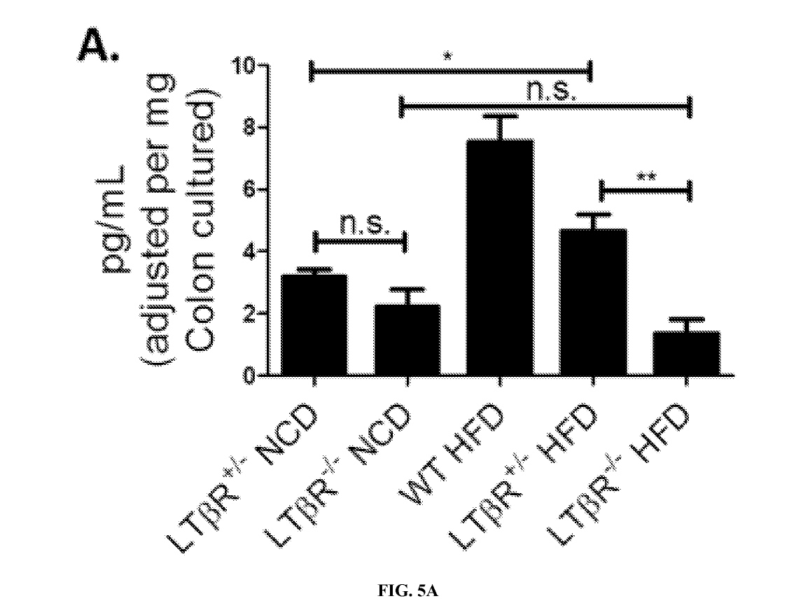

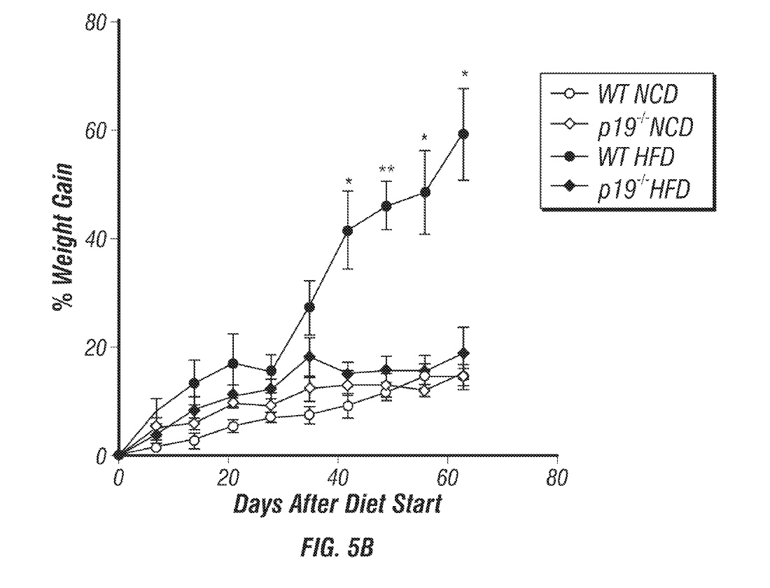

In order to confirm the importance of the LT-signaling pathway in IL-23 production, the inventor cultured colons of WT, LTβR^{+/-}, and LTβR^{-/-} animals after HFD and measured IL-23p40 in the supernatants by ELISA. The inventor observed that there was no difference in IL-23 expression between LTβR^{+/-} and LTβR^{-/-} groups on NCD; however, the inventor Observed that IL-23 was induced in LTβR^{+/-} animals after HFD but this induction did not occur in LTβR^{-/-} animals fed HFD (FIG. 5A). This is an intriguing observation because LTβR has previously been shown to impact IL-23 production in models of C. rodentium infection but not in the naïve state (Ota, 2011; Tumanov, 2011). This suggests that similar to mucosal pathogenic challenge, HFD stimulus was sufficient to evoke an immune response dependent on LTβR, which resulted in expression. In order to address the significance of IL-23 in weight gain, the inventor challenged p19^{-/-} animals with HFD; p19^{-/-} animals resisted HFD induced weight gain and excess adiposity (FIGS. 5B-D). Because HFD induced IL-23 expression is dependent on LTβR, the phenotype of p19^{-/-} animals is consistent with the necessity of the LT-pathway in controlling IL-23 for DIO.

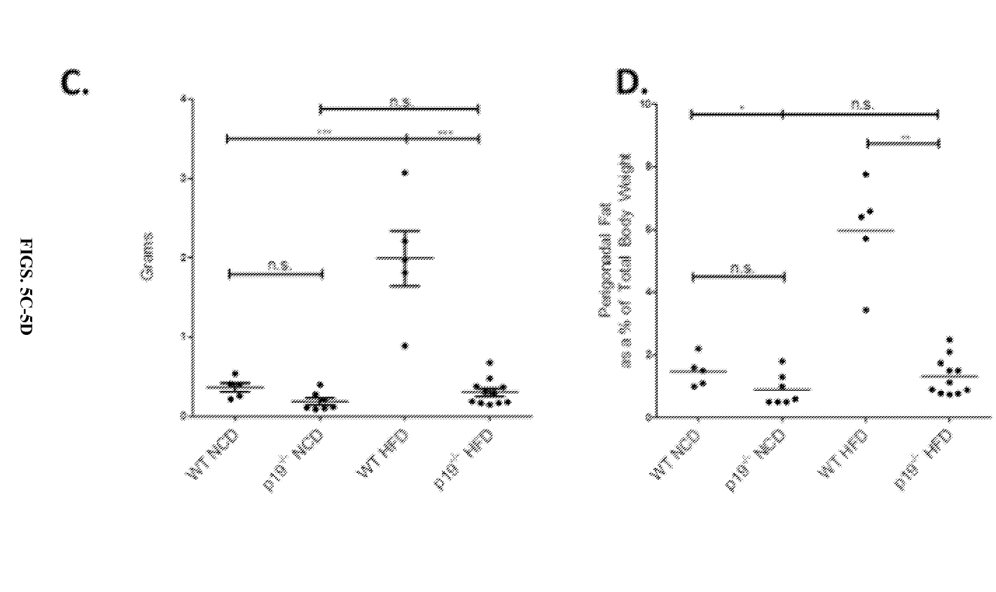

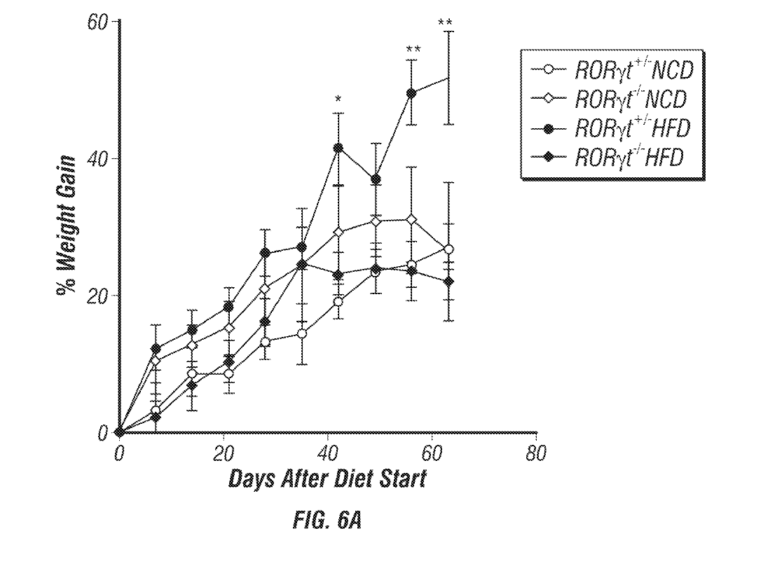

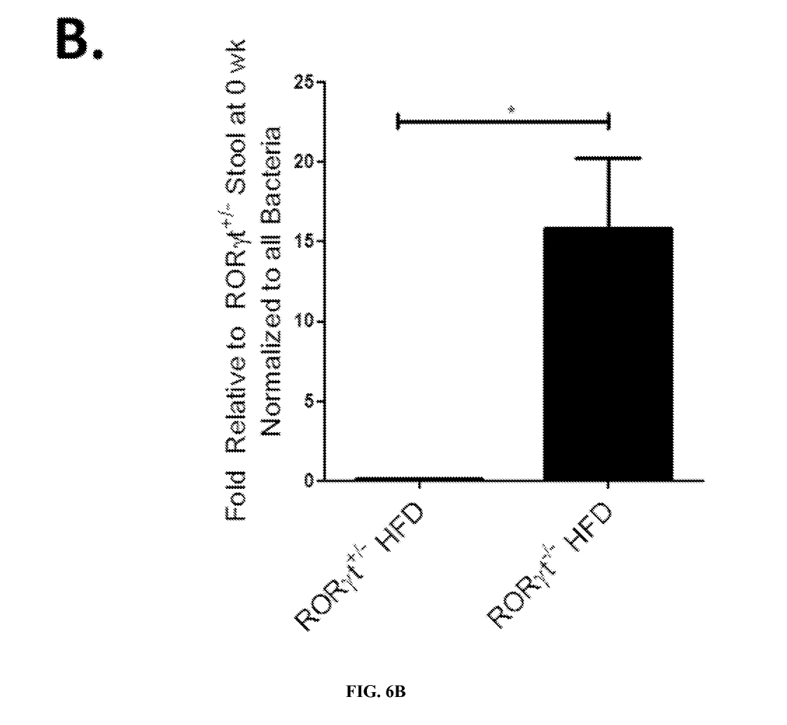

RORγt+ cells are essential for weight gain after HFD. The LT pathway is essential to enable RORγt+ innate lymphoid cells to produce IL-22 after acute bacterial infection (Ota, 2011; Tumanov, 2011). To study whether the IL-22 regulated by the LT pathway after HFD is essential for DIO, RORγt^{-/-} mice were selected because it has previously been shown that LTβR^{-/-} mice fail to evoke IL-22 production from RORγt^{+/-} lymphocytes in response to acute bacterial infection²⁵. RORγt^{-/-} mice were challenged with HFD. RORγt^{+/-} mice gained significantly greater weight after HFD than their RORγt^{-/-} littermates (FIG. 6a). It is important to note that IL-23 p19/p40 levels were similar from colons of RORγt^{+/-} and RORγt^{-/-} mice (data not shown), Which argues that the absence of RORγt+ cells does not influence IL-23 expression even though LTβR regulates IL-23. The LT-IL-23 axis is known to be essential in regulating IL-22 production from innate RORγt+ cells, and the results of RORγt-deficient mice are consistent with the involvement of this LT-mediated axis in DIO.

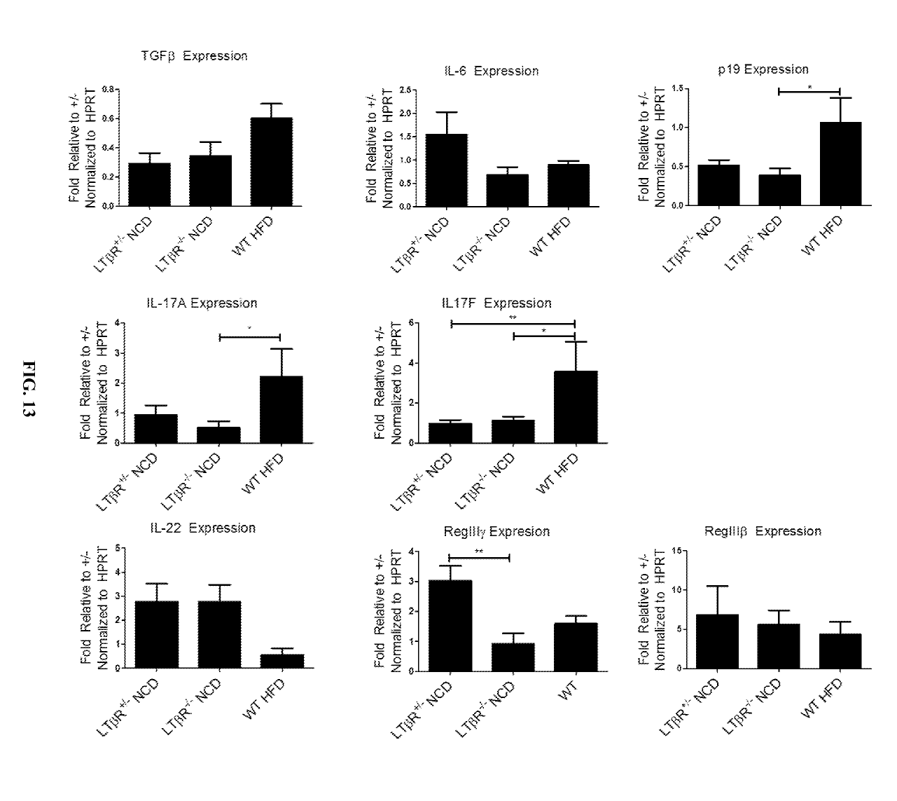

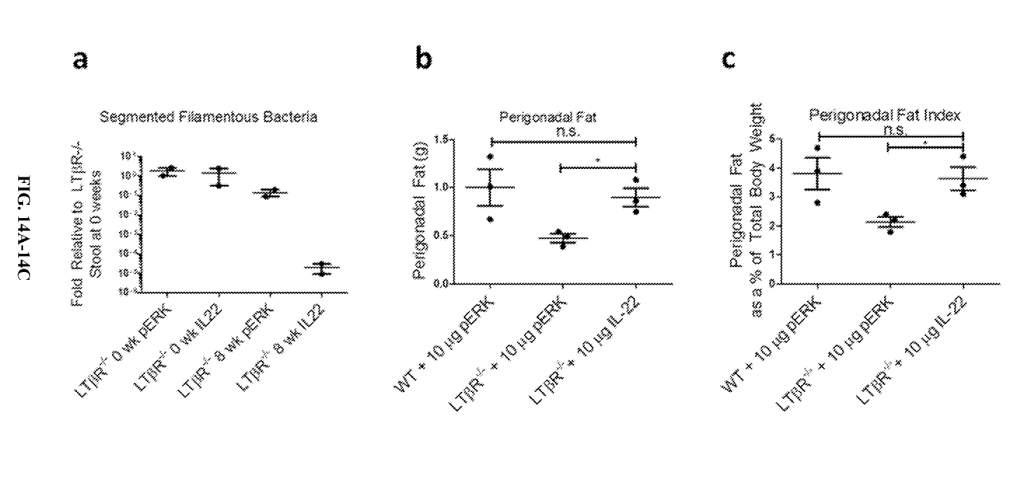

Consistent with the results in LTβR^{-/-} animals, RORγt^{-/-} animals also sustained an overgrowth of SFB after LIED (FIG. 6B). This suggests that the upstream defects in immunity are leading to a consistent downstream regulation in the microbiota. Similar to LTβR^{-/-} animals, in the absence of RORγt+ cells the perigonadal fat pad did not expand as induced by HFD (FIGS. 6C-D), Furthermore, the inventor restored IL-22 in LTβR−/− animals by hydrodynamic delivery and observed a rescue in perigonadal fat depot expansion and SFB clearance (FIG. 14). Therefore, the inventor proposes that HFD relies on a UT driven IL-23/IL-22 dependent immune response that results in the production of antimicrobial peptides and regulates specific commensal changes in the intestinal microbiota which promote DIO.

### Example 3

### Discussion

While diet appears to influence the microbiota independently of host genotype (Muegge, 2011), the possibility that innate immune responses serve as a critical pivot for species specific responses to HFD provides a potential link between host responses to diet, the intestinal microbiota, and obesity. This study demonstrates that the LT/IL23/IL-22 pathway, essential for innate immune defense against gut pathogens, is also essential for regulation of specific commensal responses to HFD. Inflammation induced by HFD is not restricted solely to adipose tissue. This was initially hinted by the observation that HFD can induce NF-κB expression in the colon early after the start of HFD (Ding, 2010). Given the important symbiosis shared between the intestinal microbiota and mucosal inflammatory responses, it is logical and important to consider how changes in immunity influence the microbiota and in turn, how those changes to the microbiota feedback to influence not only local immunity but systemic host health.

Even though the inventor reports on the importance of the innate LT-IL-23-IL22 signaling axis in DIG, the regulation of obesity by immunity is likely complex. In contrast to LT, TLR5^{-/-} mice exhibit a unique form of obesity that is also dependent on the microbiota but functions mechanistically through changes in feeding behavior; (Vijay-Kumar, 2010) the LT-pathway influences weight gain through changes in the microbiota in animals with similar feeding behavior. The inventor hypothesizes that this occurs in his model because innate lymphoid cells increase production of IL-22 in response to IL-23 induced by HFD; IL-23 itself could be agonized through another MyD88 dependent mechanism—namely IL-1 signaling or even another TLR besides TLR5. It is clear that animals fed a HFD that become obese are chronically inflamed, but more work needs to be done in order to assess whether and Which distinct immune responses after various diets are associated with distinct changes in the intestinal microbiota that lead to increased or decreased obesity. It remains to be determined whether other innate pathways might also shape the gut flora to influence energy uptake and fat deposition.

Even though some reports argue that genes play a large role in obesity (Stunkard, 1986a; Stunkard 1986b), the consistent dysbiosis present in obese individuals suggests a strong role for environmental contribution to this disease⁸. This study demonstrates the importance of a LT-dependent host immune response in DIG, but that the importance of this immune response on weight gain can be subverted by changes in. The inventor has a model system where genetic susceptibility to obesity is dependent on downstream changes to the intestinal microbiota; when hosts lack these genetic elements, they do not gain weight in response to HFD because they cannot convert their microbiota to one that promotes obesity. However, environmental exposure to LT-sufficient hosts that do successfully regulate their microbiota confers these changes in the microbiota and the weight gain phenotype to LT deficient animals. The inventor feels that the viewpoints regarding the importance of genetics and environmental importance are not at odds when it comes to obesity. The inventor proposes the possibility that the host response induced by HFD may actually help provide inertia for the obese state by facilitating occupation of microbiota that enhance energy uptake from the energy dense diet; the intestinal microbiota can thus serve as agents to transmit and infect other hosts that may not be exposed to similar diets or lack the genetic elements to promote formation of such microbiota; from this perspective, the microbiota would facilitate more efficient utilization of scarce food resources.

Population-wide implications for this argument are very exciting because this model suggests a potential to eliminate or reduce exposure to microbiota that convey metabolic disease in hosts that lack genetic predisposition or dietary exposure either through use of antibiotics or vaccination, which might reduce incidence of this pandemic illness. Even so, the precise microbes that promote such weight gain and the specific host responses that foster their growth need to be better established. Avenues of study that remain open to exploration is how HFD actually stimulates an immune response, how and which innate cells sense HFD, and how the immune response stimulated by HFD might help edit niche-occupation for members of the distal gut microbial community and promote weight gain or even type II diabetes.

All of the methods disclosed and claimed herein can be made and executed without undue experimentation in light of the present disclosure. While the methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references to the extent that they provide exemplary procedural or other details supplementary to those set forth herein, are specifically incorporated herein by reference.
Bäckhed, et al., F., Proceedings of the National Academy of Sciences. 104:979-984, 2007.
Barman, M. et al. Infect. Immun. 76:907-915, 2008.
Cella, M. et al. Nature. 457: 722-725, 2009.
Centers for Disease Control and Prevention, <world-wide-web at .edc.gov/obesity/data/trends.html.#State> (2010).
Ding, S. et al. PLoS ONE, 5:e12191, 2010.
Eberl, Mucosal Immunol, 2010.
Faith, et al., Science. 333:101-104, 2011.
Firm, Blood. 107:619-627, 2006.
Regal, et al., JAMA: The Journal of the American Medical Association 303: 235-241, 2010.
Fu & Chaplin, Annual Review of Immunology. 17: 399-433, 2003.
Ivanov, et al., Cell. 139:485-498, 2009.
Ivanov, I. I. et al. Cell. 126:1121-1133, 2006.
Klaasen, H. L. et al. Infect. Immun. 61:303-306, 1993.
Lathrop, S. K. et al. Nature 478: 250-254, 2011.
Mahajan, A. et al. Journal of Molecular Medicine. 88:515-522, 2010.
Muegge, B. D. et al. Science. 332:970-974, 2011.
Norman, et al. Journal of Clinical investigation, 96:158-162, 1995.
Ota, N. et al. Nat Immunol. 12: 941-948, 2011.
Porovko, V. et al. PLoS ONE 5:e12459, 2010.
Prakash, T. et al. Cell Host & Microbe 10:273-284, 2011.
Savage, Annual Review of Microbiology. 31: 107-133, 1977.
Schloss, P. D. et al. Appl. Environ. 75:7537-7541. 2009.
Sczesnak, A. et al. Cell Host & Microbe 10: 260-272, 2011.
Stunkard, A. J. et al, New England journal of Medicine 314:193-198, 1986.
Stunkard, JAMA: The Journal of the American Medical Association 256:51.54, 1986.
Tumanov, Alexei V. et al. Cell Host & Microbe 10:44-53, 2011.
Turnbaugh, P. J. et al. Nature 457:480-484, 2009.
Turnbaugh, P. J. et al. Nature 444:1027-1131, 2006.
Uysal, Nature 389: 610-614, 1997.
Vijay-Kumar, M. et al. Science 328:228-231, 2010.
Wang, Obesity 16:2323-2330, 2008.
White, PLoS Comput Biol 5:e1000352, 2009.
Wu, H.-J. et al. Immunity 32:815-827, 2010.
Zhao & Fernald, Comput Biol 12: 1047-1064, 2005.
Zheng, Y. et al. Nat Med 14: 282-289, 2008.

1. A method of stabilizing or reducing weight in a subject in need thereof comprising orally administering to the subject an agent that inhibits lymphotoxin, IL-22 and/or IL-23 expression and/or function in an amount sufficient to stabilize or reduce the subject's weight.

2. The method of claim 1, where the subject has excess body fat.

3. The method of claim 1, where the subject is overweight.

4. The method of claim 1, where the subject's body mass index (BMI) is from 25 kg/m² to 30 kg/m².

5. The method of claim 1, where the subject is obese or exhibits one of more symptoms of obesity.

6. The method of claim 5, where the obesity is class I.

7. The method of claim 1, where the subject's BMI is from 30 kg m² to 35 kg/m².

8. The method of claim 5, where the obesity is class II.

9. The method of claim 1, where the subject's BMI is from 35 kg/m² to 40 kg/m².

10. The method of claim 5, where the obesity is class III.

11. The method of claim 1, where the subject's BMI is from 40 kg/m² to 80 kg/m².

12. The method of any one of claims 1-11, wherein the subject is a human subject.

13. The method of any one of claims 1-13, further comprising feeding said subject a low fat and/or low calorie diet.

14. The method of claim 1, wherein the agent is a lymphotoxin inhibitor.

15. The method of claim 14, wherein at the lymphotoxin inhibitor is a small molecule, and antibody, a peptide, or a nucleic acid.

16. The method of claim 1, wherein the agent is an IL-22 inhibitor.

17. The method of claim 16, wherein at the IL-22 inhibitor is a small molecule, and antibody, a peptide, or a nucleic acid.

18. The method of claim 1, wherein the agent is an IL-23 inhibitor.

19. The method of claim 18, wherein at the IL-23 inhibitor is a small molecule, and antibody, a peptide, or a nucleic acid.

20. The method of claim 15, 17 or 18, wherein the peptide comprises an inactive fragment of lymphotoxin, IL-22 or IL-23, or an inactive fragment of a lymphotoxin receptor, a IL-22 receptor or a IL-23 receptor.

21. The method of claim 15, 17 or 18, wherein the antibody binds to a domain on lymphotoxin, IL-22 or IL-23 that interacts with the cognate receptor.

22. The method of claim 15, 17 or 18, wherein the nucleic acid is an single-stranded or double-stranded inhibitory oligonucleotide for lymphotoxin, IL-22 or IL-23.

23. The method of any one of claims 1-22, where the agent is administered daily.

24. The method of any one of claims 1-22, where the agent is formulated as a probiotic foodstuff.

25. The method of any one of claims 1-24, where the weight of the subject has been measured or will be measured.

26. The method of claim 25, where the weight of the subject has been measured prior to administering the agent and will be measured after administering the agent.

27. The method of any one of claims 1-24, where the BMI of the subject has been measured or will be measured.

28. The method of claim 27, where the BMI of the subject has been measured prior to administering the agent and will be measured after administering the agent.

29. The method of claim 1, further comprising assessing lymphotoxin and/or IL-22 and/or IL-23 expression or levels in a sample from said subject.

30. The method of claim 29, wherein said sample is a stool sample.

31. A method of preventing or inhibiting weight gain in a subject in need thereof comprising orally administering to the subject an agent that inhibits lymphotoxin, IL-22 and/or IL-23 expression and/or function in an amount sufficient to prevent or inhibit and increase in the subject's weight.

32. The method of claim 31, wherein the subject is a human subject.

33. The method of claim 31, further comprising feeding said subject a low fat and/or low calorie diet.

34. The method of claim 31, wherein the agent is a lymphotoxin inhibitor.

35. The method of claim 34, wherein at the lymphotoxin inhibitor is a small molecule, and antibody, a peptide, or a nucleic acid.

36. The method of claim 31, Wherein the agent is an IL-22 inhibitor.

37. The method of claim 36, wherein at the IL-22 inhibitor is a small molecule, and antibody, a peptide, or a nucleic acid.

38. The method of claim 31, wherein the agent is an IL-23 inhibitor.

39. The method of claim 38, wherein at the IL-23 inhibitor is a small molecule, and antibody, a peptide, or a nucleic acid.

40. The method of claim 37, 39 or 41, wherein the peptide comprises an inactive fragment of lymphotoxin, IL-22 or IL-23, or an inactive fragment of a lymphotoxin receptor, a IL-22 receptor or a IL-23 receptor.

41. The method of claim 37, 39 or 41, wherein the antibody binds to a domain on lymphotoxin, IL-22 or IL-23 that interacts with the cognate receptor.

42. The method of claim 37, 39 or 41, wherein the nucleic acid is an single-stranded or double-stranded inhibitory oligonucleotide for lymphotoxin, IL-22 or IL-23.

43. The method of claim 31, where the agent is administered daily.

44. The method of claim 31, where the agent is formulated as a probiotic foodstuff.

45. The method of claim 31, wherein said subject has a familial history of obesity.


SEQUENCE LISTING (36 sequences)

SEQ ID NO: 1 - DNA, 28 bp, Artificial Sequence
  Synthetic Primer
tgaagagctactgtaatgatcagtcaac

SEQ ID NO: 2 - DNA, 22 bp, Artificial Sequence
  Synthetic Primer
agcaagcttgcaaccttaacca

SEQ ID NO: 3 - DNA, 18 bp, Artificial Sequence
  Synthetic Primer
ggtggctcagggaaatgt

SEQ ID NO: 4 - DNA, 20 bp, Artificial Sequence
  Synthetic Primer
gacagagcaggcaggtacag

SEQ ID NO: 5 - DNA, 18 bp, Artificial Sequence
  Synthetic Primer
cactgatacgcctgagtg

SEQ ID NO: 6 - DNA, 18 bp, Artificial Sequence
  Synthetic Primer
gtgagcgctgaatcgaaa

SEQ ID NO: 7 - DNA, 24 bp, Artificial Sequence
  Synthetic Primer
tccaatgctctcctaacagataag

SEQ ID NO: 8 - DNA, 22 bp, Artificial Sequence
  Synthetic Primer
caagatgaattggatggtcttg

SEQ ID NO: 9 - DNA, 22 bp, Artificial Sequence
  Synthetic Primer
ctccagaaggccctcagactac

SEQ ID NO: 10 - DNA, 24 bp, Artificial Sequence
  Synthetic Primer
agctttccctccgcattgacacag

SEQ ID NO: 11 - DNA, 24 bp, Artificial Sequence
  Synthetic Primer
gaggataacactgtgagagttgac

SEQ ID NO: 12 - DNA, 21 bp, Artificial Sequence
  Synthetic Primer
gagttcatggtgctgtcttcc

SEQ ID NO: 13 - DNA, 20 bp, Artificial Sequence
  Synthetic Primer
tccgaggagtcagtgctaaa

SEQ ID NO: 14 - DNA, 20 bp, Artificial Sequence
  Synthetic Primer
agaacgtcttccagggtgaa

SEQ ID NO: 15 - DNA, 20 bp, Artificial Sequence
  Synthetic Primer
atggctcctattgctatgcc

SEQ ID NO: 16 - DNA, 19 bp, Artificial Sequence
  Synthetic Primer
gatgtcctgagggcctctt

SEQ ID NO: 17 - DNA, 20 bp, Artificial Sequence
  Synthetic Primer
atggctcctactgctatgcc

SEQ ID NO: 18 - DNA, 19 bp, Artificial Sequence
  Synthetic Primer
gtgtcctccaggcctcttt

SEQ ID NO: 19 - DNA, 21 bp, Artificial Sequence
  Synthetic Primer
actcctacgggaggcagcagt

SEQ ID NO: 20 - DNA, 18 bp, Artificial Sequence
  Synthetic Primer
attaccgcggctgctggc

SEQ ID NO: 21 - DNA, 21 bp, Artificial Sequence
  Synthetic Primer
gacgctgaggcatgagagcat

SEQ ID NO: 22 - DNA, 21 bp, Artificial Sequence
  Synthetic Primer
gacggcacggattgttattca

SEQ ID NO: 23 - DNA, 55 bp, Artificial Sequence
  Synthetic Primer
cgtatcgcctccctcgcgccatcagacgagtgcgtagagtttgatcctggctcag

SEQ ID NO: 24 - DNA, 55 bp, Artificial Sequence
  Synthetic Primer
cgtatcgcctccctcgcgccatcagacgctcgacaagagtttgatcctggctcag

SEQ ID NO: 25 - DNA, 55 bp, Artificial Sequence
  Synthetic Primer
cgtatcgcctccctcgcgccatcagagacgcactcagagtttgatcctggctcag

SEQ ID NO: 26 - DNA, 55 bp, Artificial Sequence
  Synthetic Primer
cgtatcgcctccctcgcgccatcagagcactgtagagagtttgatcctggctcag

SEQ ID NO: 27 - DNA, 55 bp, Artificial Sequence
  Synthetic Primer
cgtatcgcctccctcgcgccatcagatcagacacgagagtttgatcctggctcag

SEQ ID NO: 28 - DNA, 55 bp, Artificial Sequence
  Synthetic Primer
cgtatcgcctccctcgcgccatcagatatcgcgagagagtttgatcctggctcag

SEQ ID NO: 29 - DNA, 55 bp, Artificial Sequence
  Synthetic Primer
cgtatcgcctccctcgcgccatcagcgtgtctctaagagtttgatcctggctcag

SEQ ID NO: 30 - DNA, 55 bp, Artificial Sequence
  Synthetic Primer
cgtatcgcctccctcgcgccatcagctcgcgtgtcagagtttgatcctggctcag

SEQ ID NO: 31 - DNA, 55 bp, Artificial Sequence
  Synthetic Primer
cgtatcgcctccctcgcgccatcagtagtatcagcagagtttgatcctggctcag

SEQ ID NO: 32 - DNA, 55 bp, Artificial Sequence
  Synthetic Primer
cgtatcgcctccctcgcgccatcagtctctatgcgagagtttgatcctggctcag

SEQ ID NO: 33 - DNA, 55 bp, Artificial Sequence
  Synthetic Primer
cgtatcgcctccctcgcgccatcagtgatacgtctagagtttgatcctggctcag

SEQ ID NO: 34 - DNA, 55 bp, Artificial Sequence
  Synthetic Primer
cgtatcgcctccctcgcgccatcagcatagtagtgagagtttgatcctggctcag

SEQ ID NO: 35 - DNA, 55 bp, Artificial Sequence
  Synthetic Primer
cgtatcgcctccctcgcgccatcagcgagagatacagagtttgatcctggctcag

SEQ ID NO: 36 - DNA, 44 bp, Artificial Sequence
  Synthetic Primer
ctatgcgccttgccagcccgctcagtgctgcctcccgtaggagt